(19) United States
(12) Patent Application Publication
(10) Pub. No.: US 2023/0296633 A1
(43) Pub. Date: Sep. 21, 2023

(54) METHODS AND SYSTEMS FOR DETECTION OF FIBRIN FORMATION OR REMOVAL AT THE NANO-SCALE

(71) Applicants: Unchained Labs, Pleasanton, CA (US); Trustees of Boston University, Boston, MA (US)

(72) Inventors: John Connor, Newton, MA (US); Shinichiro Kurosawa, Waban, MA (US); Deborah Stearns-Kurosawa, Waban, MA (US); George G. Daaboul, Watertown, MA (US)

(21) Appl. No.: 17/431,584

(22) PCT Filed: Feb. 28, 2020
(86) PCT No.: PCT/US2020/020336

§ 371 Date: Aug. 17, 2021

Related U.S. Application Data
(60) Provisional application No. 62/812,696, filed on Mar. 1, 2019.

(51) Int. Cl. G01N 33/86; G01N 21/64

(52) U.S. Cl. CPC G01N 33/86; G01N 21/6428; G01N 21/6456; G01N 2021/6439; G01N 2333/75

(57) ABSTRACT

Systems and methods for imaging and tracking fibrin formation via interaction of a test sample with a clotting agent or for imaging and tracking fibrin removal by an anti-clotting agent are described.In certain embodiments, the systems (200) comprise a planar reflective substrate (222, 224) comprising one or more capture agents and/or one or more fibrin reference regions; a mount for holding the substrate; an illumination light source (201) for directing illumination light toward a top surface of the substrate with fibrin (226)formed thereon; an image detector (232, 234) aligned with respect to the mount for detecting a portion of the illumination light that is scattered by the fibrin, and/or reflected by the reflective substrate, thereby obtaining a label-free image of fibrin formation or fibrin removal; a processor of a computing device (240); and a memory having instructions stored thereon, wherein the instructions, when executed by the processor, cause the processor to: receive and/or access data corresponding to the one or more label free images, and use the one or more label-free images to determine one or more measures of fibrin formation or fibrin removal.

### CROSS-REFERENCE TO RELATED APPLICATIONS

This application claims priority to and benefit of U.S. Application Serial No. 62/812,696, filed on Mar. 1, 2019, the contents of which are incorporated by reference in their entirety.

### TECHNICAL FIELD

This invention relates generally to compositions, systems, and methods for the assessment of blood clotting and fibrin formation on a substrate. More particularly, in certain embodiments, the invention relates to methods for the visualization of fibrin formation using a microscopy approach based on interference reflectance (e.g., for clinical applications).

### GOVERNMENT SUPPORT STATEMENT

This invention was made with Government Support under Contract Nos. AI096159 and AI102931 awarded by the National Institutes of Health. The Government has certain rights in the invention.

### BACKGROUND

The ability to identify a patient’s propensity to form insoluble fibers from proteins is critical to the medical field in assessing health and disease. For example, in certain instances, the patient’s ability to form insoluble protein fibers is important to natural biological processes (e.g., blood clotting) and in other instances, diseases may result from the malformation of these proteins (e.g., Alzheimer’s disease) and/or the improper coagulation of fibers (e.g., stroke).

The blood clotting cascade is an example of a biological process that is important to asses. Deficiencies in this cascade or the structure of fibrinogen (a key component of blood clots) can lead to severe health issues. An important step of the clotting cascade is the conversion of fibrinogen, a globular protein monomer, to fibrin, a polymer. Fibrin is a fibrous, insoluble protein that comprises a key component of blood clots. Assessment of an ability to form blood clots is an important step in many medical applications such as, without limitation, in assessment of surgical risks, identification of potential health complications in cancer, control of diseases (e.g., hemophilia), assessment of risks during sepsis, and assessments of risks during infection with various diseases.

Currently, assays (e.g., thromboelastography, e.g., thromboelastometry) exist that are used to track clotting ability of a patient’s sample. Such assays are used to assess potential risks and aid in the diagnosis of various diseases. These tests are used clinically to track clotting propensity under various testing conditions.

Other conventional clinical tests can assess thrombin generation in samples. Thrombin is an important part of the clotting cascade and converts fibrinogen to fibrin. Accordingly, the assessment of thrombin generation tests help to assess a patient’s ability to form clots. Thrombin generation tests include optical path interruption tests, such as measurements of the activated partial thromboplastin time (aPTT) and prothrombin time (PT).

Clotting ability may also be assessed with fibrin-formation tests. However, fibrin-formation tests require incorporation of fluorescent tracers. Moreover, fibrin-formation assays (e.g., those measuring clotting mechanics) are limited to identifying properties of bulk clotting in whole blood samples and do not provide direct information on individual fiber formation.

Sophisticated, highly specialized microscopy techniques (e.g., electron microscopy, total internal reflection fluorescence (TIRF) microscopy, confocal microscopy) are required to observe the formation of individual fibers. However, observing fiber formation requires large instruments that are extremely costly, which precludes their use outside of specialized facilities and locales. In addition, specialized reagents, such as fluorescent tags, present an additional cost for some of these techniques (e.g., TIRF and confocal microscopy) in order to visualize the protein forming fibers.

Therefore, conventional fibrin formation assays are limited as they do not allow for easy, in-clinic, portable assessments of clotting (e.g., as at surgical suites or remotely in doctors’ offices) or other sites where the assessment is most needed and in a rapid manner. Moreover, such techniques do not directly provide information on individual fiber formation, which is important for understanding a patient’s propensity to form improper clots either through clotting too slowly (e.g., as in patients utilizing Warfarin) or too quickly (e.g., as in patients at risk for stroke).

Accordingly, there exists a need for methods, compositions, and systems that can provide for the assessment of clotting through the analysis of fibrin fiber formation.

### SUMMARY

Presented herein are compositions, systems, and methods related to directly visualizing fiber formation at the nano-scale using a “label-free” (e.g., lacking a tagged fluorophore), light-microscopy approach for the direct assessment of fiber formation. Unlike conventional assays, which do not provide information on individual fiber formation, the technology described herein can track formation of individual fibrils and allow for monitoring of the conditions under which they form. Additionally, the technology described herein can monitor the efficacy of a variety of clot-busting therapies (e.g., Warfarin) on patient samples, thereby identifying a broad range of potential new treatments of diseases implicated in fibril formation.

In certain embodiments, the disclosed light-microscopy platform allows for the evaluation of fibrin or amyloid fibril formation on the surface of a reflective substrate. In certain embodiments, the optical substrate comprises a stack of thin, transparent dielectric layers that is designed for both specific scattering enhancement at a first target wavelength and fluorescence enhancement at a second target wavelength. The ability of the described optical substrates to simultaneously co-localize both enhanced contrast and fluorescence signals provides for increased sensitivity and detection of nanofibers (e.g., fibrin fibers). This substrate allows for the direct visualization of features, such as individual fibrin fibers, without the use of fluorescent labeling. In certain embodiments, fibrin fibers are formed on top of a substrate which has been pre-spotted with protein (e.g., fibrinogen), which serves as a nucleating protein for the formation of fibrin fibers by capturing a nucleator (e.g., thrombin) on the surface. Therefore, when a sample (e.g., from a subject) containing fibrinogen (the monomeric precursor to fibrin) is placed in contact with the surface, fibrin fibers are formed on the substrate in a short amount of time. The formation of the fibers can be evaluated through direct imaging to provide clinically relevant information in a time period of 5 minutes or less with only a few reagents.

In one aspect, the invention is directed to a method of imaging and tracking fibrin formation (e.g., natural fibrin formation; e.g., synthetic fibrin formation) via interaction of a test sample with a clotting agent (e.g., an immobilized clotting agent), the method comprising: (a) contacting the test sample [e.g., a sample obtained from a subject; e.g., a complex biological sample, such as blood, plasma, saliva, etc.; e.g., a processed sample (e.g., comprising fibrinogen in a buffer)] with the clotting agent [e.g., a thrombin (e.g., thrombin, e.g., alpha-thrombin); e.g., calcium; e.g., activated platelets; e.g., divalent cations][e.g., so as to provide for testing of the test sample, e.g., based on the interaction of the test sample with the captured clotting agent (e.g., for normal clotting; e.g., for abnormal clotting; e.g., based on imaging of formation of fibers); e.g., so as to provide for testing of the test sample and/or one or more additional compounds by first initiating clot formation via interaction of the test sample with the clotting agent, and then contacting the test sample with the one or more additional compounds]; (b) contacting a top surface of a substrate (e.g., a substantially planar reflective substrate) with the (i) the test sample and/or (ii) the clotting agent [e.g., contacting the top surface of the substrate with the test sample prior contacting the test sample with the clotting agent, and then contacting the top surface of the substrate with the clotting agent; e.g., contacting the top surface of the substrate with the clotting agent, and then with the test sample; e.g., contacting the test sample with the clotting agent (e.g., in solution), and then contacting the top surface of the substrate with the test sample and clotting agent], thereby providing for formation of fibrin at the top surface of the substrate {e.g., via capture of at least one of (i) one or more components of the test sample, (ii) the clotting agent, and (iii) one or more product components resulting from interaction of the test sample with the clotting agent [e.g., wherein the surface of the substrate comprises one or more primary capture agents (e.g., fibrinogen), each primary capture agent specific to at least one of (i) the one or more components of the test sample, (ii) the clotting agent, and (iii) the one or more product components]}; (c) directing illumination light toward the top surface of the substrate, thereby illuminating the top surface of the substrate along with the fibrin formed thereon (e.g., due to an interaction of the captured clotting agent with the test sample); (d) detecting, with one or more imaging detectors, a label-free scattering signal corresponding to a portion of the illumination light that is (A) scattered by the fibrin, and/or (B) reflected by the reflective substrate, thereby obtaining one or more label-free images of fibrin formation [e.g., wherein the illumination light that is (A) scattered by the fibrin interferes with the illumination light that is (B) reflected by the substrate at the one or more detectors, thereby enhancing contrast of the fibrin in the one or more label free images]; and (e) using the one or more label-free images to determine (e.g., by a processor of a computing device) one or more measures of fibrin formation [e.g., one or more static measures (e.g., average density, mass, branching, cross-linking, a number of fibers formed, a measure of fiber length, a measure of fiber contrast; a measure fiber width, etc.), e.g., associated with a point in time and/or label-free image; e.g., one or more time-dependent measures (e.g., such as a rate of change, time to reaching a particular value, etc., of any of one or more static measures), e.g., determined from two or more label-free images collected at two or more different times] [e.g., and using the one or more measures of fibrin formation to determine one or more prognostic values (e.g., indicative of thrombotic risk, clotting characteristics, disease state (e.g., deep vein thrombosis; e.g., a genetic defects, etc.) for the test sample and/or a subject associated with the test sample (e.g., from whom the test sample was obtained)].

In certain embodiments, the test sample comprises cancer plasma (e.g., plasma from a patient with cancer).

In certain embodiments, the test sample is obtained from a subject {e.g., a human subject [e.g., wherein the test sample comprises human plasma (e.g., the human plasma comprising an anticoagulant); e.g., an animal subject} {e.g., undergoing treatment (e.g., undergoing surgery, preparing for surgery, recovering from surgery), diagnosed as having a particular condition and/or defect [e.g., a genetic defect (e.g., in a clotting pathway)], and/or thought to be at risk for a particular condition (e.g., deep vein thrombosis, pre-eclampsia, stroke, etc.)}.

In certain embodiments, the top surface of the substrate comprises one or more primary capture agents [e.g., fibrinogen, e.g., collagen; e.g., a capture agent (e.g., an antibody) specific to fibrinogen], each specific to at least one of (i) the one or more components of the test sample, (ii) the clotting agent, and (iii) the one or more product components.

In certain embodiments, the method comprises drying the top surface of the substrate (e.g., and washing with solution prior to the drying down; e.g., and contacting the top surface of the substrate with a crosslinking agent, thereby fixing (crosslinking) the fibrin formed thereon) following step (b) (e.g., following step (a); e.g., before performing step (a)) and before performing step (c) [e.g., and storing the assay in a stable, dried down form, e.g., for at least an hour (e.g., for at least 6 hours; e.g., for at least 12 hours; e.g., for at least 24 hours; e.g., for at least one month or more) after step (b) and prior to step (c)] (e.g., such that steps (c) and (d) may be performed later by a central lab facility).

In certain embodiments, step (b) comprises incubating the clotting agent with the test sample for a duration of about 5 minutes or less (e.g., for a duration of about 3 to 5 minutes).

In certain embodiments, the method comprises performing steps (c) and (d) at one or more time points after contacting the clotting agent with the test sample (e.g., at one or more times within about 60 minutes after first contacting the clotting agent with the test sample), so as to obtain one or more label-free images of fibrin formation, each corresponding to a particular time point after the contacting the clotting agent with the test sample. In certain embodiments, the method comprises performing steps (c) and (d) at a plurality of time points while incubating the clotting agent with the test sample, thereby obtaining a plurality of images tracking formation of fibrin. In certain embodiments, the method comprises performing steps (c) and (d) at one or more times prior to and/or at the same time as step (b), thereby obtaining one or more reference images of the top surface of the substrate prior to formation of fibrin following the contacting the clotting agent with the test sample.

In certain embodiments, step (d) comprises imaging the top surface of the substrate and/or any fibers formed thereon at a resolution better than 600 nm (e.g., better than 500 nm; e.g., better than 450 nm)(e.g., using a microscope objective with a numerical aperture above 0.5 (e.g., above 0.6; e.g., approximately 0.7 or greater)[e.g., using a wavelength ranging from about 300 to 700 nm (e.g., ranging from about 400 to 500 nm)](e.g., sufficient to distinguish between individual fibers).

In certain embodiments, the one or more measures of fibrin formation comprise one or more members selected from the group consisting of: a number of fibers [e.g., an total number of fibers within one or more predefined regions (e.g., a spot) on the top surface of the substrate that comprises the clotting agent]; a density of fibers (e.g., a number per unit area, a ratio of area occupied by fibrin to total area, etc.)[e.g., within one or more predefined regions (e.g., a spot) on the top surface of the substrate that comprises the clotting agent]; a measure of fiber length [e.g., an average, a maximum, a median, a minimum, etc., length of fiber, e.g., within one or more predefined regions (e.g., a spot) on the top surface of the substrate that comprises the clotting agent]; a measure of fiber thickness [e.g., an average, a maximum, a median, a minimum, etc., thickness of fibers, e.g., within one or more predefined regions (e.g., a spot) on the top surface of the substrate that comprises the clotting agent]; a measure of branching capacity; a measure of fibrin cross-linking; and a measure of contrast [e.g., an average, a maximum, a median, a minimum, etc., fiber contrast, e.g., within one or more predefined regions (e.g., a spot) on the top surface of the substrate that comprises the clotting agent].

In certain embodiments, wherein step (e) comprises: identifying, within at least a portion of the one or more label-free images, one or more point spread functions each corresponding to a piece of fibrin having a sub-diffraction limited length; determining, for each of the one or more point spread functions, a contrast value, thereby determining one or more contrast values; and using the one or more determined contrast values (e.g., for each of the one or more point spread functions) to determine a length of the corresponding piece of fibrin.

In certain embodiments, the method comprises performing steps (c) and (d) at a plurality of time points while incubating the clotting agent with the test sample, thereby obtaining a plurality of images tracking formation of fibrin, and using the plurality of images to determine one or more time-dependent measures of fibrin formation.

In certain embodiments, the method comprises using the one or more measures of fibrin formation to determine one or more prognostic values [e.g., a value representing a risk of having and/or developing a particular disease (e.g., a thrombotic risk, risk of having and/or developing deep vein thrombosis, likelihood of having a particular genetic defect, etc.); e.g., a value representing a particular disease state (e.g., positive or negative for a particular disease, such as deep vein thrombosis; e.g., a genetic defect); e.g., a value representing a particular clotting characteristic] for the test sample and/or a subject associated with the test sample (e.g., from whom the test sample was obtained).

In certain embodiments, the one or more prognostic values comprise an activated partial thromboplastin time (APPT) and/or a prothrombin time (PT)(e.g., a length of time it takes for fibrin bundles to form as compared to a known value).

In certain embodiments, the one or more prognostic values comprises a relative risk of one or more particular diseases and/or conditions (e.g., stroke).

In certain embodiments, the test sample is obtained from a patient having a condition (e.g., selected from the group consisting of disseminated intravascular coagulation (DIC), trauma induced coagulopathy, cancer-associated coagulopathy, deep vein thrombosis, hypercoagulable states, thromboembolism, stroke).

In certain embodiments, the one or more fibrin forming components is/are fluorescently labeled [e.g., and the method comprises detecting fluorescent light emitted by the one or more fluorescently labeled fibrin forming components to obtain one or more fluorescence images of fibrin formation].

In certain embodiments, a component other than fibrin/fibrinogen is attached to the assay chip.

In certain embodiments, wherein the top surface of the substrate comprises one or more secondary capture agents (e.g., antibodies), each specific to one or more disease-associated biomolecules (e.g., virus, viral nucleic acid, etc.), each disease-associated biomolecule associated with a particular infectious disease, thereby providing for testing the test sample for the particular infectious disease.

In certain embodiments, fibrin-capturing molecules are used to assess the presence of micro-clots within a plasma or blood sample.

In certain embodiments, step (a) comprises contacting the test sample with an unknown clotting agent (e.g., a small molecule, a biologics or bio-similar, etc.), thereby providing for testing the unknown clotting agent for potential to induce clotting and/or prevent clotting.

In certain embodiments, the top surface of the substrate comprises (e.g., is coated with) a material under test (e.g., a stent material and/or coating) (e.g., thereby providing for assessing the ability of these elements to resist fibrin binding).

In certain embodiments, the method further comprises contacting the test sample with one or more (e.g., known; e.g., unknown) secondary agents (e.g., a small molecule, a biologics or biosimilars, etc.), thereby providing for assessing the influence of the one or more secondary agents on clotting (e.g., inducing and/or preventing) and/or assessing removal of clots via the one or more secondary agents. In certain embodiments, the one or more secondary agents comprise anti-clotting agents [e.g., blockers of thrombin activation (e.g., thrombomodulin); e.g., modifiers of plasminogen activity such as PAI1 or other serpin molecules; e.g., agents such as warfarin, and factor X inhibitors]. In certain embodiments, the one or more secondary agents comprise one or more clot promoting agents (e.g., anomalous clot promoting agents such as infection associated promoters of clotting, e.g., bacterial lipopolysaccharide). In certain embodiments, the test sample is a known reference sample.

In certain embodiments, the clotting agent is a known reference clotting agent (e.g., having been previously characterized with respect to a population of known test samples; e.g., so as to provide for assessing clotting in the test sample).

In another aspect, the invention is directed to a method of imaging and tracking fibrin removal by an anti-clotting agent (e.g., a drug), the method comprising: (a) contacting a top surface of a substrate (e.g., a substantially planar reflective substrate) with an anti-clotting buffer comprising an anti-clotting agent [e.g., an enzyme, (e.g., plasmin), a drug (e.g., one with an unknown effect on clotting)] [e.g., blockers of thrombin activation (e.g., thrombomodulin); e.g., modifiers of plasminogen activity such as PAI1 or other serpin molecules; e.g., agents such as warfarin, and factor X inhibitors], wherein the top surface of the substrate comprises one or more fibrin reference regions each comprising a known (e.g., previously characterized) fibrin layer [e.g., so as to provide for testing of the anti-clotting agent; e.g., based on the interaction of the test sample with the anti-clotting agent (e.g., for normal clotting; e.g., for abnormal clotting; e.g., based on imaging of formation of fibers)]; (b) directing illumination light toward at least a portion of the one or more fibrin reference regions of the top surface of the substrate, thereby illuminating the top surface of the substrate along with fibrin formed thereon within the portion of the fibrin reference regions (e.g., due to an interaction of the captured clotting agent with the test sample; e.g., due to an interaction of the anti-clotting agent with the test sample); (c) detecting, with one or more imaging detectors, a label-free scattering signal corresponding to a portion of the illumination light that is (A) scattered the fibrin, and/or (B) reflected by the reflective substrate, thereby obtaining one or more label-free images of fibrin formation; and (d) using the detected label-free scattering signal to determine one or more measures of fibrin formation (e.g., average density, mass, branching, etc.) for the test sample (e.g., and using the one or more measures of fibrin formation to determine one or more prognostic values indicative of thrombotic risk, clotting characteristics, disease state, within the patient).

In certain embodiments, the method comprises performing steps (b) and (c) at one or more time points after contacting the top surface of the substrate with the anti-clotting buffer, so as to obtain one or more label-free images of fibrin formation, each corresponding to a particular time point after the contacting the top surface of the substrate with the anti-clotting agent.

In certain embodiments, the method comprises performing steps (b) and (c) at a plurality of time points while incubating the anti-clotting buffer the top surface of the substrate, thereby obtaining a plurality of images tracking removal of fibrin formation by the anti-clotting buffer.

In certain embodiments, the method comprises performing steps (b) and (c) at one or more times prior to and/or at the same time as step (a), thereby obtaining one or more reference images of the top surface of the substrate prior to removal of fibrin formation following the contacting the top surface of the substrate with the anti-clotting buffer.

In certain embodiments, step (c) comprises imaging the top surface of the substrate and/or any fibers formed thereon at a resolution better than 600 nm (e.g., better than 500 nm; e.g., better than 450 nm)(e.g., using a microscope objective with a numerical aperture above 0.5 (e.g., above 0.6; e.g., approximately 0.7 or greater) [e.g., using a wavelength ranging from about 300 to 700 nm (e.g., ranging from about 400 to 500 nm)].

In certain embodiments, the one or more measures of fibrin formation comprise one or more members selected from the group consisting of: a number of fibers [e.g., an total number of fibers within one or more of the fibrin reference regions]; a density of fibers (e.g., a number per unit area, a ratio of area occupied by fibrin to total area, etc.)[e.g., within one or more of the fibrin reference regions]; a measure of fiber length [e.g., an average, a maximum, a median, a minimum, etc., length of fiber, e.g., within one or more of the fibrin reference regions]; a measure of fiber thickness [e.g., an average, a maximum, a median, a minimum, etc., thickness of fibers, e.g., within one or more of the fibrin reference regions]; a measure of branching capacity; a measure of fibrin cross-linking; and a measure of contrast [e.g., an average, a maximum, a median, a minimum, etc., fiber contrast, e.g., within one or more of the fibrin reference regions].

In certain embodiments, step (e) comprises: identifying, within at least a portion of the one or more label-free images, one or more point spread functions each corresponding to a piece of fibrin having a sub-diffraction limited length; determining, for each of the one or more point spread functions, a contrast value, thereby determining one or more contrast values; and using the one or more determined contrast values (e.g., for each of the one or more point spread functions) to determine a length of the corresponding piece of fibrin.

In certain embodiments, the method comprises performing steps (b) and (c) at a plurality of time points while incubating the anti-clotting buffer with the top surface of the substrate, thereby obtaining a plurality of images tracking removal of fibrin formation, and using the plurality of images to determine one or more time-dependent measures of fibrin formation.

In certain embodiments, the one or more fibrin forming components is/are fluorescently labeled [e.g., and the method comprises detecting fluorescent light emitted by the one or more fluorescently labeled fibrin forming components to obtain one or more fluorescence images of fibrin formation].

In certain embodiments, a component other than fibrin/fibrinogen is attached to the assay chip.

In another aspect, the invention is directed toward a method of generating fibrin formation on a surface of a substrate (e.g., a substantially planar substrate) for chip-based testing via immobilized clotting agents (e.g., thrombin), the method comprising: (a) contacting a top surface of the substrate (e.g., a substantially planar reflective substrate) with a clotting buffer comprising a clotting agent [e.g., a thrombin (e.g., thrombin, e.g., alpha-thrombin); e.g., calcium], wherein the top surface of the substrate comprises one or more capture agents (e.g., fibrinogen, e.g., collagen), each capture agent specific to the particular clotting agent (e.g., thrombin), thereby capturing the clotting agent onto the top surface of the substrate; (b) following step (a), contacting the clotting agent with a test sample [e.g., a sample obtained from a subject; e.g., a complex biological sample, such as blood, plasma, saliva, etc.; e.g., a processed sample (e.g., comprising fibrinogen in a buffer)][e.g., so as to provide for testing of the test sample; e.g., based on the interaction of the test sample with the captured clotting agent (e.g., for normal clotting; e.g., for abnormal clotting; e.g., based on imaging of formation of fibers)].

In certain embodiments, the method comprises drying down the chip for stable storage and/or further testing.

In certain embodiments, the method has one or more of the features articulated in paragraphs [0015] to [0036].

In another aspect, the method is directed to a pre-spotted planar reflective substrate (e.g., comprising a sub-micron thickness silicon dioxide layer on top of a silicon layer), wherein a top surface of the pre-spotted planar reflective substrate comprises a plurality of capture agent spots, each capture agent spot comprising a particular capture agent specific to a particular clotting agent and/or component of fibrin.

In certain embodiments, the method has one or more of the features articulated in paragraphs [0015] to [0036].

In another aspect, the invention is directed towards fibrin reference planar reflective substrate (e.g., comprising a sub-micron thickness silicon dioxide layer on top of a silicon layer), wherein a top surface of the fibrin reference substrate comprises a plurality of fibrin reference regions each comprising a known (e.g., previously characterized) fibrin layer [e.g., so as to provide for testing of an anti-clotting agent; e.g., based on the interaction of the anti-clotting agent with the fibrin reference region (e.g., to test for removal of fibrin)].

In certain embodiments, the method has one or more of the features articulated in paragraphs [0015] to [0036].

In another aspect, the method is directed towards a system for imaging and tracking fibrin formation (e.g., natural fibrin formation; e.g., synthetic fibrin formation) via interaction of a test sample with a clotting agent (e.g., an immobilized clotting agent), the system comprising: (a) a planar reflective substrate (e.g., comprising a sub-micron thickness silicon dioxide layer on top of a silicon layer) comprising one or more capture agents (e.g., immobilized on the substrate surface) and/or one or more fibrin reference regions; (b) a mount for holding a substrate; (c) one or more illumination light sources aligned with respect to the mount so as to and direct illumination light toward a top surface of the substrate (e.g., when held by the mount), so as to provide for illumination of the top surface of the substrate along with fibrin formed thereon; (d) one or more detectors aligned with respect to the mount and operable to detect a portion of the illumination light that is (A) scattered by the fibrin, and/or (B) reflected by the reflective substrate, thereby providing for obtaining one or more label-free images of fibrin formation [e.g., wherein the illumination light that is (A) scattered by the fibrin interferes with the illumination light that is (B) reflected by the substrate at the one or more detectors, thereby enhancing contrast of the fibrin in the one or more label free images]; (e) a processor of a computing device; and (f) a memory having instructions stored thereon, wherein the instructions, when executed by the processor, cause the processor to: receive and/or access data corresponding to the one or more label free images; and use the one or more label-free images to determine (e.g., by a processor of a computing device) one or more measures of fibrin formation.

In certain embodiments, the top surface of the planar reflective substrate comprises a plurality of capture agent spots, each capture agent spot comprising a particular capture agent specific to a particular clotting agent and/or component of fibrin.

In certain embodiments, the top surface of the planar reflective substrate comprises a plurality of fibrin reference regions each comprising a known (e.g., previously characterized) fibrin layer [e.g., so as to provide for testing of an anti-clotting agent; e.g., based on the interaction of the anti-clotting agent with the fibrin reference region (e.g., to test for removal of fibrin)].

In certain embodiments, the system comprises an objective lens aligned to (i) collect light (A) scattered by the fibrin and/or reflected by the reflective substrate and (ii) direct the collected light onto the one or more detectors. In certain embodiments, the objective lens has a numerical aperture above 0.5 (e.g., above 0.6; e.g., approximately 0.7 or greater). In certain embodiments, the objective lens has a magnification ranging from about 4X to about 100X.

In another aspect, the invention is directed to a method of imaging and tracking fibril formation (e.g., natural fibrin formation; e.g., synthetic fibrin formation; e.g., amyloid fibril) via interaction of a test sample with a nucleation agent (e.g., an agent that induces fibril formation; e.g., an immobilized clotting agent), the method comprising: (a) contacting the test sample [e.g., a sample obtained from a subject; e.g., a complex biological sample, such as blood, plasma, saliva, cerebrospinal fluid (CSF), etc.; e.g., a processed sample (e.g., comprising fibrinogen in a buffer)] with the nucleation agent {e.g., a clotting agent; e.g., an amyloidosis precursor protein (e.g., to induce formation of amyloid fibrils)} [e.g., so as to provide for testing of the test sample, e.g., based on the interaction of the test sample with the nucleation agent; e.g., so as to provide for testing of the test sample and/or one or more additional compounds by first initiating fibril formation via interaction of the test sample with the nucleation agent, and then contacting the test sample with the one or more additional compounds]; (b) contacting a top surface of a substrate (e.g., a substantially planar reflective substrate) with the (i) the test sample and/or (ii) the nucleation agent [e.g., contacting the top surface of the substrate with the test sample prior contacting the test sample with the nucleation agent, and then contacting the top surface of the substrate with the nucleation agent; e.g., contacting the top surface of the substrate with the nucleation agent, and then with the test sample; e.g., contacting the test sample with the nucleation agent (e.g., in solution), and then contacting the top surface of the substrate with the test sample and nucleation agent], thereby providing for formation of one or more fibrils at the top surface of the substrate {e.g., via capture of at least one of (i) one or more components of the test sample, (ii) the nucleation agent, and (iii) one or more product components resulting from interaction of the test sample with the nucleation agent [e.g., wherein the surface of the substrate comprises one or more primary capture agents (e.g., fibrinogen), each primary capture agent specific to at least one of (i) the one or more components of the test sample, (ii) the nucleation agent, and (iii) the one or more product components]}; (c) directing illumination light toward the top surface of the substrate, thereby illuminating the top surface of the substrate along with the one or more fibrils formed thereon (e.g., due to an interaction of the captured clotting agent with the test sample); (d) detecting, with one or more imaging detectors, a label-free scattering signal corresponding to a portion of the illumination light that is (A) scattered by the one or more fibrils, and/or (B) reflected by the reflective substrate, thereby obtaining one or more label-free images of fibril formation [e.g., wherein the illumination light that is (A) scattered by the one or more fibrils interferes with the illumination light that is (B) reflected by the substrate at the one or more detectors, thereby enhancing contrast of the one or more fibrils in the one or more label free images]; and (e) using the one or more label-free images to determine (e.g., by a processor of a computing device) one or more measures of fibril formation [e.g., one or more static measures (e.g., average density, mass, branching, cross-linking, a number of fibers formed, a measure of fiber length, a measure of fibril contrast; a measure fiber width, etc.), e.g., associated with a point in time and/or label-free image; e.g., one or more time-dependent measures (e.g., such as a rate of change, time to reaching a particular value, etc., of any of one or more static measures), e.g., determined from two or more label-free images collected at two or more different times] {e.g., and using the one or more measures of fibril formation to determine one or more prognostic values [e.g., indicative of disease state and/or risk for the test sample and/or a subject associated with the test sample (e.g., from whom the test sample was obtained)]} .

In certain embodiments, the one or more fibrils correspond to amyloid fibrils and the nucleation agent comprises an amyloidosis precursor protein [e.g., an amyloidosis precursor protein selected from the group consisting of: Amyloid precursor protein (e.g., associated with Alzheimer’s disease; e.g., wherein the amyloid fibril comprises (e.g., is formed of) Aβ peptides); Atrial natriuretic factor (ANF) (e.g., associated with Atrial amyloidosis; e.g., wherein the amyloid fibril comprises (e.g., is formed of) Amyloid ANF); Prion protein (PrPc) (e.g., associated with Spongiform encephalopathies; e.g., wherein the amyloid fibril comprises (e.g., is formed of) PrPsc); Immunoglobulin light and heavy chains (e.g., Primary systemic amyloidosis; e.g., wherein the amyloid fibril comprises (e.g., is formed of) AL and/or AH); Wild-type transthyretin (e.g., associated with Senile systemic amyloidosis; e.g., wherein the amyloid fibril comprises (e.g., is formed of) ATTR); β2-microglobulin (e.g., associated with Hemodialysis-related amyloidosis; e.g., wherein the amyloid fibril comprises (e.g., is formed of) Aβ2M); Lysozyme (e.g., associated with Hereditary nonneuropathic systemic amyloidosis; e.g., wherein the amyloid fibril comprises (e.g., is formed of) ALys); Pro-IAPP (e.g., associated with Type II diabetes; e.g., wherein the amyloid fibril comprises (e.g., is formed of) IAPP and/or “amylin”); Insulin (e.g., associated with Injection-localized amyloidosis; e.g., wherein the amyloid fibril comprises (e.g., is formed of) AIns); (Apo) serum amyloid A (e.g., associated with Secondary systemic amyloidosis; e.g., wherein the amyloid fibril comprises (e.g., is formed of) Serum amyloid A); Cystatin C (e.g., associated with Hereditary cerebral amyloid angiopathy; e.g., wherein the amyloid fibril comprises (e.g., is formed of) ACys); Gelsolin (e.g., associated with Finnish hereditary systemic amyloidosis; e.g., wherein the amyloid fibril comprises (e.g., is formed of) AGel); Transthyretin variants (e.g., associated with Familial amyloid polyneuropathy I; e.g., wherein the amyloid fibril comprises (e.g., is formed of) ATTR); Apolipoprotein A1 (e.g., associated with Familial amyloid polyneuropathy II; e.g., wherein the amyloid fibril comprises (e.g., is formed of) AApoA1); Prolactin (e.g., associated with Ageing pituitary, prolactinomas; e.g., wherein the amyloid fibril comprises (e.g., is formed of) APro); Fibrinogen αA-chain (e.g., associated with Familial amyloidosis; e.g., wherein the amyloid fibril comprises (e.g., is formed of) AFib); and Amyloid Bri Precursor Protein (e.g., associated with British familial dementia; e.g., wherein the amyloid fibril comprises (e.g., is formed of) ABri)].

In certain embodiments, at least one of the one or more fibrils is and/or comprises amyloid fibril and the method further comprises contacting the test sample with one or more test compounds {e.g., a small molecule, a biologics or biosimilars, etc. [e.g., to assess efficacy of the one or more test compounds to remove (e.g., dissolve) and/or prevent formation of the amyloid fibril (e.g., and thereby treat disease, e.g., Alzheimer’s)]; e.g., a compound that enhances and/or stabilizes fibril formation (e.g., to study what causes and/or exasperates the disease)}.

Elements of embodiments involving one aspect of the invention (e.g., compositions, e.g., systems, e.g., methods) can be applied in embodiments involving other aspects of the invention, and vice versa.

### BRIEF DESCRIPTION OF THE FIGURES

The foregoing and other objects, aspects, features, and advantages of the present disclosure will become more apparent and better understood by referring to the following description taken in conjunction with the accompanying drawings, in which:

FIG. 1 is schematic diagram of fibrin assembly, branching, lateral fibril association, and γ-chain cross-linking, according to an illustrative embodiment.

FIG. 2 is a diagram of an exemplary system for use in imaging e.g., of fibers bound to a substrate, according to an illustrative embodiment of the present disclosure.

FIG. 3A is an illustration showing an instrument for imaging substrates as described herein, e.g., for detection of fibrils, according to an illustrative embodiment.

FIG. 3B is an illustration of a reflective chip (substrate), as described herein, according to an illustrative embodiment.

FIG. 3C is an illustration of a reflective chip disposed within a microfluidic cassette, which allows flowing of a sample over the substrate, according to an illustrative embodiment.

FIG. 3D is an illustration of an array of samples and/or agents (e.g., capture agents, nucleation agents, and/or clotting agents) on a substrate, as described herein, according to an illustrative embodiment.

FIG. 4 is a block diagram of method 400 of imaging and tracking fibrin formation via interaction of a test sample with a clotting agent, according to an illustrative embodiment.

FIG. 5 is a block diagram of method 500 of imaging and tracking fibrin removal by an anti-clotting agent, according to an illustrative embodiment.

FIG. 6 is a block diagram of a method 600 of generating fibrin formation on a surface of a substrate for chip-based testing via immobilized clotting agents, according to an illustrative embodiment.

FIG. 7 is an illustrative representation of a device 700 whereon the top surface of the substrate 705, a capture agent 710 is present.

FIG. 8 is a block diagram of a method 800 of imaging and tracking fibril formation via interaction of a test sample with a nucleation agent.

FIG. 9A is a series of images showing spots of a pre-scanned chip spotted with decreasing concentrations of fibrinogen. The concentrations decrease from left to right.

FIG. 9B is a series of images of the same chip spots of FIG. 9A showing the same spots after incubation with a high concentration of fibrinogen for 3 min.

FIG. 10A is a series of images showing spots of a pre-scanned chip spotted with decreasing concentrations of fibrinogen (from left to right).

FIG. 10B is a series of images of the same chip of FIG. 10A after incubation with a high concentration of fibrinogen for 10 min.

FIG. 11A is the same image of FIG. 10B.

FIG. 11B is a series of images of the same chip spots of FIG. 10B after incubation with plasmin.

FIG. 12A is a series of images showing spots of a pre-scanned chip spotted with decreasing concentrations of fibrinogen. The concentration of fibrinogen decreases from left to right.

FIG. 12B is an image of the same chip of FIG. 12A after incubation with thrombin, followed by a 10 minute incubation with tris-buffered saline with 1% gelatin (TBSG).

FIG. 13A is an image showing spots of a pre-scanned chip that has been spotted with decreasing concentrations of fibrinogen. The concentration of fibrinogen decreases from left to right.

FIG. 13B is an image of FIG. 13A after the chip has been incubated with TBSG, followed by a 10 min incubation of a high concentration of fibrinogen.

FIG. 14 is a block diagram of an exemplary cloud computing environment, used in certain embodiments.

FIG. 15 is a block diagram of an example computing device and an example mobile computing device used in certain embodiments.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements. Drawings are presented herein for illustration purposes, not for limitation.

### DEFINITIONS

In order for the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The articles “a” and “an” are used herein to refer to one or to more than one (i.e., at least one) of the grammatical object of the article. By way of example, “an element” means one element or more than one element. Thus, in this specification and the appended claims, the singular forms “a,” “an,” and “the” include plural references unless the context clearly dictates otherwise. Thus, for example, reference to a pharmaceutical composition comprising “an agent” includes reference to two or more agents.

“Antigen-binding site” or “binding portion”: The term “antigen-binding site” or “binding portion” refers to the part of the immunoglobulin (Ig) molecule that participates in antigen binding. The antigen binding site is formed by amino acid residues of the N-terminal variable (“V”) regions of the heavy (“H”) and light (“L”) chains. Three highly divergent stretches within the variable regions of the heavy and light chains, referred to as hypervariable regions, are interposed between more conserved flanking stretches known as “framework regions,” or “FRs”. The term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

“Capture Agent”: As described herein, the term “capture agent” refers to any entity that binds to a target of interest as described herein. In many embodiments, a capture agent of interest is one that binds specifically with its target in that it discriminates its target from other potential binding partners in a particular interaction contact. In general, a capture agent may be or comprise an entity of any chemical class (e.g., polymer, non-polymer, small molecule, polypeptide, carbohydrate, lipid, nucleic acid, etc.). In some embodiments, a capture agent is a single chemical entity. In some embodiments, a capture agent is a complex of two or more discrete chemical entities associated with one another under relevant conditions by non-covalent interactions. For example, those skilled in the art will appreciate that in some embodiments, a capture agent may comprise a “generic” binding moiety (e.g., one of biotin/avidin/streptavidin and/or a class-specific antibody) and a “specific” binding moiety (e.g., an antibody or aptamers with a particular molecular target) that is linked to the partner of the generic biding moiety. In some embodiments, such an approach can permit modular assembly of multiple capture agents through linkage of different specific binding moieties with the same generic binding moiety partner. In some embodiments, capture agents are or comprise peptides and/or polypeptides (including, e.g., antibodies or antibody fragments). In certain embodiments, the peptides and/or polypeptides may be further labeled with an isotope. In some embodiments, capture agents are or comprise antibodies (e.g., including monoclonal antibodies, polyclonal antibodies, bispecific antibodies, or antigen-binding fragments thereof, and antibody fragment including, ScFv, F(ab), F(ab′)2, Fv). In some embodiments, capture agents are or comprise small molecules. In some embodiments, capture agents are or comprise nucleic acids. In some embodiments, capture agents are aptamers. In some embodiments, capture agents are polymers. In some embodiments, capture agents are non-polymeric in that they lack polymeric moieties. In some embodiments, binding agents are or comprise carbohydrates. In certain embodiments, capture agents are or comprise nucleic acids, such as DNA or RNA. In certain embodiments as described herein, a capture agent may be present on the top surface of a substrate as described herein (e.g., an optical substrate, e.g., a reflective substrate). In certain embodiments, a capture agent may be specific to at least one of (i) the one or more components of the test sample (e.g., a capture agent specific to fibrinogen), (ii) the clotting agent, and (iii) the one or more product components. In certain embodiments, capture agents are fibrinogen and/or collagen.

“Biocompatible”: The term “biocompatible”, as used herein is intended to describe materials that do not elicit a substantial detrimental response in vivo. In certain embodiments, the materials are “biocompatible” if they are not toxic to cells. In certain embodiments, materials are “biocompatible” if their addition to cells in vitro results in less than or equal to 20% cell death, and/or their administration in vivo does not induce inflammation or other such adverse effects. In certain embodiments, materials are biodegradable.

“Branching capacity”: In certain embodiments, the term “branching capacity” refers the propensity of linear fibrils forming branches. The amount of branching along a given fibril length and the complexity of the branching network can be termed branching capacity. In certain embodiments, “branching capacity” refers to the propensity of associated fibrils to diverge and form separate branches. In certain embodiments, “branching capacity” refers to the propensity to converge into single branch.

“Contrast”: The term “contrast” when referring to a nanoparticle or other biological structure (e.g., a fibril, a fiber) bound to an optical substrate refers to the total scattered intensity of the particle or structure over the intensity of the background, or reflectivity of the substrate.

“Electromagnetic radiation”, “radiation”: As used herein, the terms “electromagnetic radiation” and “radiation” is understood to mean self-propagating waves in space of electric and magnetic components that oscillate at right angles to each other and to the direction of propagation, and are in phase with each other. Electromagnetic radiation includes: radio waves, microwaves, red, infrared, and near-infrared light, visible light, ultraviolet light, X-rays and gamma rays.

“Image”: The term “image”, as used herein, is understood to mean a visual display or any data representation that may be interpreted for visual display. For example, a three-dimensional image may include a dataset of values of a given quantity that varies in three spatial dimensions. A three-dimensional image (e.g., a three-dimensional data representation) may be displayed in two-dimensions (e.g., on a two-dimensional screen, or on a two-dimensional printout). In certain embodiments, the term “image” may refer to, for example, to a multi-dimensional image (e.g., a multi-dimensional (e.g., four dimensional) data representation) that is displayed in two-dimensions (e.g., on a two-dimensional screen, or on a two-dimensional printout). The term “image” may refer, for example, to an optical image, an x-ray image, an image generated by: positron emission tomography (PET), magnetic resonance, (MR) single photon emission computed tomography (SPECT), and/or ultrasound, and any combination of these.

“Sample” or “biological sample“: A sample refers to any sample containing a biomolecular target, such as, for example, blood, plasma, serum, gastrointestinal secretions, homogenates of tissues or tumors, synovial fluid, feces, saliva, sputum, cyst fluid, amniotic fluid, cerebrospinal fluid, peritoneal fluid, lung lavage fluid, semen, lymphatic fluid, tears, prostatic fluid, or cellular lysates. A sample may also be obtained from an environmental source, such as water sample obtained from a polluted lake or other body of water, or a liquid sample obtained from a food source believed to contaminated. As used herein the terms “sample” or “biological sample” means any sample, including, but not limited to cells, organisms, lysed cells, cellular extracts, nuclear extracts, components of cells or organisms, extracellular fluid, media in which cells are cultured, blood, plasma, serum, gastrointestinal secretions, homogenates of tissues or tumors, synovial fluid, feces, saliva, sputum, cyst fluid, amniotic fluid, cerebrospinal fluid, peritoneal fluid, lung lavage fluid, semen, lymphatic fluid, tears and prostatic fluid. In addition, a sample can be a viral or bacterial sample, a sample obtained from an environmental source, such as a body of polluted water, an air sample, or a soil sample, as well as a food industry sample.

“Subject”: As used herein, the term “subject” includes humans and mammals (e.g., mice, rats, pigs, cats, dogs, and horses). In many embodiments, subjects are mammals, particularly primates, especially humans. In certain embodiments, subjects are livestock such as cattle, sheep, goats, cows, swine, and the like; poultry such as chickens, ducks, geese, turkeys, and the like; and domesticated animals particularly pets such as dogs and cats. In certain embodiments (e.g., particularly in research contexts) subject mammals will be, for example, rodents (e.g., mice, rats, hamsters), rabbits, primates, or swine such as inbred pigs and the like.

“Label” or “tag”: The terms “label” or “tag”, as used herein, refer to a composition capable of producing a detectable signal indicative of the presence of the target in an assay sample. Suitable labels include radioisotopes, nucleotide chromophores, enzymes, molecular substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means.

“Sensor”, “Detector”: As used herein, the terms “sensor” and “detector” are used interchangeably and include any sensor of electromagnetic radiation including, but not limited to, CCD camera, CMOS camera, intensified CCD (I-CCD) camera, Electron-Multiplication CCD (EM-CCD) camera, Electron-Bombardment CCD (EB-CCD) camera, scientific CMOS (sCMOS) camera, photomultiplier tubes, photodiodes, and avalanche photodiodes.

“Substrate”: As used herein, the term “substrate” refers to a substrate that is reflective for specific excitation wavelengths (e.g., one or more excitation wavelengths). In certain embodiments, the substrate is or comprises an optical substrate. In certain embodiments, the substrate is an optical substrate that enhances a fluorescence signal emitted by a fluorophore. In certain embodiment, the substrate is an optical substrate that enhances “contrast” signal (or “label-free” signal) that comprises scattered signal intensity over substrate reflectivity at a non-fluorescent wavelength. In certain embodiments, the substrate is an optical substrate that simultaneously (1) enhances a fluorescence signal emitted by a fluorophore and (2) enhances “contrast” signal (or “label-free” signal) that comprises scattered signal intensity over substrate reflectivity at a non-fluorescent wavelength. In certain embodiments, the optical substrate comprises a thin, transparent, dielectric layer. In alternative embodiments, the optical substrate comprises a stack of thin, transparent dielectric layers, for example, that is designed for both specific scattering enhancement at a first target wavelength and fluorescence enhancement at a second target wavelength. In certain embodiments, the substrate is a substrate as described by PCT/US17/16434 entitled “DETECTION OF EXOSOMES HAVING SURFACE MARKERS” filed on Feb. 3, 2017, the content of which is hereby incorporated by reference in its entirety. In certain embodiments, the substrate is or comprises a substantially planar reflective substrate.

“Cancer”: As used herein, the terms “cancer,” “tumor” or “tumor tissue” refer to an abnormal mass of tissue that results from excessive cell division, in certain cases tissue comprising cells which express, over-express, or abnormally express a hyperproliferative cell protein. A cancer, tumor or tumor tissue comprises “tumor cells” which are neoplastic cells with abnormal growth properties and no useful bodily function. Cancers, tumors, tumor tissue and tumor cells may be benign or malignant. A cancer, tumor or tumor tissue may also comprise “tumor-associated non-tumor cells”, e.g., vascular cells which form blood vessels to supply the tumor or tumor tissue. Non-tumor cells may be induced to replicate and develop by tumor cells, for example, the induction of angiogenesis in a tumor or tumor tissue.

Examples of cancer include, but are not limited to, carcinoma, lymphoma, blastoma, sarcoma, and leukemia or lymphoid malignancies. More particular examples of such cancers are noted below and include: squamous cell cancer (e.g. Epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulvar cancer, thyroid cancer, hepatic carcinoma, anal carcinoma, penile carcinoma, as well as head and neck cancer. The term “cancer” includes primary malignant cells or tumors (e.g., those whose cells have not migrated to sites in the subject’s body other than the site of the original malignancy or tumor) and secondary malignant cells or tumors (e.g., those arising from metastasis, the migration of malignant cells or tumor cells to secondary sites that are different from the site of the original tumor).

In some embodiments, the cancer is an adenocarcinoma. In some embodiments, the cancer is selected from breast, lung, head or neck, prostate, esophageal, tracheal, brain, liver, bladder, stomach, pancreatic, ovarian, uterine, cervical, testicular, colon, rectal, and skin. In some embodiments the caner is an adenocarcinoma of the breast, lung, head or neck, prostate, esophagus, trachea, brain, liver, bladder, stomach, pancreas, ovary, uterus cervix, testicular, colon, rectum, or skin. In some embodiments the cancer is selected from pancreatic, lung (e.g., small cell or non-small cell), and breast.

Other examples of cancers or malignancies include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin’s Disease, Adult Hodgkin’s Lymphoma, Adult Lymphocytic Leukemia, Adult Non-Hodgkin’s Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin’s Disease, Childhood Hodgkin’s Lymphoma, Childhood Hypothalamic and Visual Pathway Glioma, Childhood Lymphoblastic Leukemia, Childhood Medulloblastoma, Childhood Non-Hodgkin’s Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing’s Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher’s Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin’s Disease, Hodgkin’s Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi’s Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin’s Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom’s Macroglobulinemia, Wilms’ Tumor, and any other hyperproliferative disease, besides neoplasia, located in an organ system listed above.

“Reflective Substrate”: As used herein, reflective substrate is used to refer to a substrate for reflecting light back to a detector. As used herein, the term “reflective substrate” is intended to encompass a variety of substrates and/or substrate materials having various reflectance. In certain embodiments, the reflective substrate comprises a single layer. In certain embodiments, a reflective substrate comprises an oxide layer on a silicon base. In certain embodiments, the reflective substrate comprises multiple layers (e.g., as described in further detail herein).

In certain embodiments, a reflective substrate has a reflectance greater than a particular minimum value at one or more wavelengths and/or spectral ranges of interest. Exemplary spectral ranges include, but are not limited to, the ultra-violet (UV) spectral range, ranging from about 400 nm to 450 nm, the blue spectral range, ranging from about 460 nm to about 500 nm, the green spectral range, ranging from about 520 nm to about 560 nm, the red spectral range, ranging from about 640 nm to about 680 nm, and the deep red spectral range, ranging from about 710 nm to about 750 nm. For example, a reflective substrate may have a “reflectance” or “reflectivity” greater than or approximately equal to 25% (e.g., greater than 30%, e.g., greater than 40%, e.g., greater than 50%, e.g., greater than 60%, greater than 70%) across one or more wavelengths and/or spectral bands of interest. In certain embodiments, the reflective substrate has reflectance greater than 80% or more across one or more wavelengths and/or spectral bands of interest.

A reflective substrate may have a reflectance that varies according to a particular functional form, such as a sinuosoid, e.g., produced by optical interference effects, such that it has a particular reflectance at one or more wavelengths and/or spectral ranges of interest, but a relatively low reflectance at other wavelengths.

“Substantially”: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

“Therapeutic agent”: As used herein, the phrase “therapeutic agent” refers to any agent that has a therapeutic effect and/or elicits a desired biological and/or pharmacological effect, when administered to a subject.

“Treatment”: As used herein, the term “treatment” (also “treat” or “treating”) refers to any administration of a substance that partially or completely alleviates, ameliorates, relives, inhibits, delays onset of, reduces severity of, and/or reduces incidence of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In certain embodiments, treatment may be of a subject who has been diagnosed as suffering from the relevant disease, disorder, and/or condition. In certain embodiments, treatment may be of a subject known to have one or more susceptibility factors that are statistically correlated with increased risk of development of the relevant disease, disorder, and/or condition.

### DETAILED DESCRIPTION

It is contemplated that systems, architectures, devices, methods, and processes of the claimed invention encompass variations and adaptations developed using information from the embodiments described herein. Adaptation and/or modification of the systems, architectures, devices, methods, and processes described herein may be performed, as contemplated by this description.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Headers are provided for the convenience of the reader - the presence and/or placement of a header is not intended to limit the scope of the subject matter described herein.

Documents are incorporated herein by reference as noted. Where there is any discrepancy in the meaning of a particular term, the meaning provided in the Definition section above is controlling.

In certain embodiments, the described methods are used in combination with the system and methods described in PCT/US17/16434 entitled “DETECTION OF EXOSOMES HAVING SURFACE MARKERS” filed on Feb. 3, 2017, the content of which is hereby incorporated by reference in its entirety.

Presented herein are compositions, systems, and methods related to the detection and measurement of fibril formation on a substrate. In certain embodiments, the technology relates to methods for the visualization of fibril formation from complex biological samples using a microscopy approach based on interference reflectance (e.g., for clinical applications). The technology allows a user to analyze the temporal (e.g., time to form, e.g., time to remove) and physical (e.g., density, thickness, branching structures) features of fibrils and/or fibrillar structures (e.g., fibers). Fibrils, as used herein, may also be used interchangeably to refer to fibers. Moreover, monitoring under what conditions the fibrils and/or fibrillar structures are formed and what compounds can disrupt their formation is an important feature. In certain embodiments, the technology further allows the users to characterize, diagnose, and/or stage disease. In certain embodiments, the technology also allows users to develop and/or identify candidate therapies for correcting of the clot formation and/or “dissolving” clots.

In certain embodiments, the light microscopy technology allows for tracking of the formation of fibrils. For example, fibrinogen monomers polymerize to form fibrils of fibrin, an insoluble protein. Fibrin is a critically important component in clotting and defects in the processes of clotting in fibrin can lead to health issues (e.g., excessive bleeding). Therefore, tracking the formation of bundles of fibrin fibrils is critical for understanding a propensity of a subject to form improper clots (e.g., stroke, e.g., Alzheimer’s disease), for understanding how effectively clot-busting therapies are working, to identify conditions under which clots are forming, and for identifying new approaches for controlling clotting.

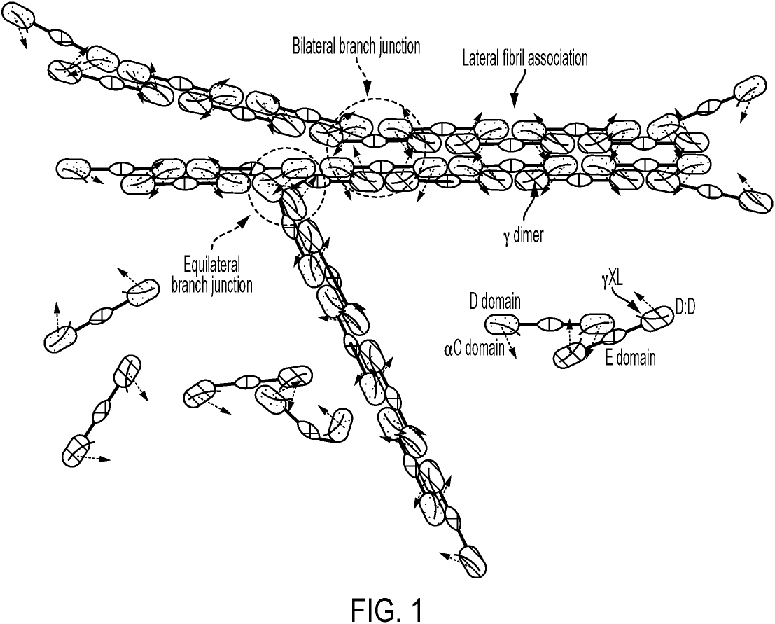

Without being bound to any particular theory, some of types of fibrin structures that fibrinogen can form are shown in FIG. 1. The schematic diagram shows fibrin assembly, branching, lateral fibril association, and γ-chain cross-linking. Fibrinogen monomers are represented in two color schemes for ease of recognition. Cross-linked γ-chains are positioned ‘transversely’ between fibril strands, as discussed in text (adapted from Mosesson, MW. (2005), Fibrinogen and fibrin structure and functions. Journal of Thrombosis and Haemostasis, 3: 1894-1904). In other structures, fibrin can interact with proteins (e.g., the amyloid-beta protein) to form amyloid fibrils in certain diseases (e.g., Alzheimer’s disease).

In certain embodiments, the technology comprises a chip-based endpoint assay system for the assessment of fibrin fiber-bundle formation in plasma or other bodily fluids. In certain embodiments, the technology comprises a cartridge-based kinetic assay where the rate and nature of fibrin fiber-bundle formation in plasma or bodily fluids is tracked over time.

In certain embodiments, the technology comprises an assay in which the thickness and branching capacity of the fibrin fiber-bundles is assessed.

In certain embodiments, the technology comprises an assay in which the propensity of blood samples to form fibrin fiber-bundles is used to stratify surgical patients for their thrombotic risk.

In certain embodiments, the technology comprises an assay to determine whether certain cancer patients are at thrombotic risk.

In certain embodiments, the technology comprises an in vitro assay system to determine the effectiveness of clot-dissolving enzymes or small molecules.

In certain embodiments, the technology comprises an assay that replaces or complements viscoelastic tests.

In certain embodiments, the technology comprises an assay that replaces or complements prothrombin time (PT) and/or activated partial thromboplastin time (aPTT) tests.

In certain embodiments, the technology comprises an assay used to track clotting in patients with disseminated intravascular coagulation (DIC), trauma induced coagulopathy, cancer-associated coagulopathy, deep vein thrombosis, hypercoagulable states, thromboembolisms, strokes, etc.

In certain embodiments, the technology comprises the monitoring of amyloid fibril formation to study the biology or test compounds as potential drugs to treat Alzheimer’s disease by breaking up established fibrils in the brain or to stop their formation.

In contrast to conventional assays, the described technology utilizes reflectance microscopy to visualize the formation of fibrin fibers on a substrate. In certain embodiments, the assay allows direct visualization of fibrin bundle assembly through use of a light-microscope setup. Prior investigations have used Electron Microscopy, TIRF, and Confocal Microscopy. Such systems are not suitable for bedside and/or point of care use.

In certain embodiments, the assays can be used at the bedside in surgical suites or remotely in doctor’s offices. Assay time can be very rapid, currently 3-5 minutes is within the assay window. Cost of goods for assay development is low, and assay can be shelf-stable.

### A. Optical Sensors and Detection Methods

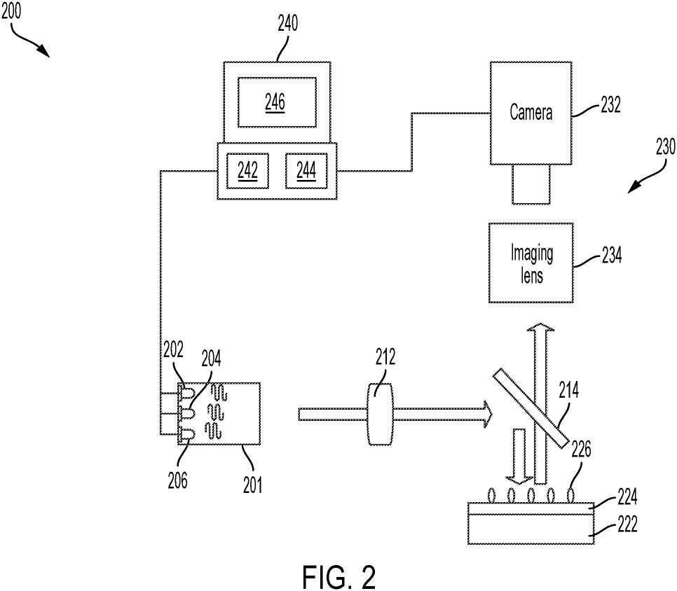

In one or more embodiments, the technology described herein includes apparatus and systems that can detect the formation of fibrils and/or fibrillar structures on the surface of a substrate. In certain embodiments, the fibrils may be fluorescently labeled using the protocols and methods described herein. FIG. 2 illustrates a diagrammatic view of an example imaging system 200 used for imaging fibrils in which the substrates described herein may be used. The system 200 can include an illumination source 201, directing and providing illumination light onto a substrate 222. In certain embodiments as depicted in FIG. 2, the substrate is a reflective substrate 222, having a single oxide layer 223 and the fibrils 226 to be detected, and an imaging system 230 for capturing images of the light reflected by the substrate 222, the oxide layer 224, and the fibrils 226. In another embodiment, the optical substrate 222 may be a multilayered reflective substrate (not shown) substantially as described herein. The multilayered reflective substrate may comprise a stack of thin, transparent dielectric layers, for example, that is designed for both specific scattering enhancement at a first target wavelength and fluorescence enhancement at a second target wavelength. In certain embodiments, the substrate is mounted and held in place using a mount suitable for the dimensions of the substrate (e.g., a microscope slide mount, a mount for a well plate).

The system 200 can also include a computer system 240 for controlling the illumination source 201 and receiving imaging signals from the imaging system 230. In an embodiment, the illumination source 201 includes incoherent light source (LED) 202 that provides incoherent light in one wavelength having a substantially narrow band of wavelengths. In an embodiment, the illumination source 201 includes a coherent light source (laser). The illumination source may also serve as an excitation source for use in fluorescently tagged fibril detection / classification applications (e.g., for the detection of fluorescent labels). In certain embodiments, multiple illumination sources may be utilized. In some embodiments, the illumination source 201 can include three or more coherent or incoherent light sources 202, 204, 206 that produce incoherent light in three different wavelengths. The Light Emitting Diodes (LEDs) or equivalent light sources, each provide incoherent light at one of the plurality of wavelengths. In some embodiments, the illumination source 201 can include an array of illumination elements, including one or more illumination elements providing light at the same wavelength and being arranged in a geometric (e.g., circular or rectangular), random, or spatially displaced array. The light from the illumination source 201 can be directed through a focusing lens 212 and other optical elements (e.g., polarizing lens, filters and light conditioning components, not shown) to a beam splitter 214 that directs the light onto the substrate 222, the oxide layer 224 and the fibrils 226. Optical components can be provided to condition the light to uniformly illuminate substantially the entire surface of the layered substrate 222. The light reflected by the substrate 222, the oxide layer 224 and the fibrils 226 can be directed through the beam splitter 214 and imaging lens 234 into a detector (e.g., a camera) 232 to capture images of the substrate surface. In certain embodiments, there may be more than one detector. In certain embodiments, the imaging lens is a high magnification and high resolution objective lens. In certain embodiments, the objective lens is a high magnification objective lens having a magnification ranging from about 4X-100X (e.g., 4X, 10X, 20X, 40X, 60X, 100X). In certain embodiments, the objective lens has a numerical aperture ranging from about 0.1 and about 1.3 (e.g., 0.13, 0.3, 0.5. 0.75, 0.85, 1.25, 1.3). In certain embodiments, light is emitted by a fluorescent label substantially attached to or co-localized with the fibrils. The camera 232 can be, for example, a CCD camera (color or monochromatic) and produce image signals representative of the image based on data corresponding to the illumination light scattered by the fibrils and/or reflected by the substrate. In another embodiment, the camera 232 can produce image signals representative of the image based on data corresponding to the detected fluorescent light emitted by the fluorescent tags attached to and/or associated with the fibrils. The image signals can be sent from the camera 232 to the computer system 210 either by a wireless or wired connection.

Computer system 240 can include one or more central processing units (CPUs) and associated memory (including volatile and non-volatile memory, such as, RAM, ROM, flash, optical and magnetic memory) and a display 246 for presenting information to a user. The memory can store one or more computer programs that can be executed by the CPUs to store and process the image data and produce images of the substrate surface. Additional computer programs can be provided for analyzing the image data and the images to detect interference patterns and the fibrils 226 on the surface of the oxide layer 224 of the substrate 222. Additional computer programs can also provide for analyzing the images of the fluorescent light in conjunction with the image of the fibrils to enhance imaging of the fibrils. In certain embodiments, one or more measures of fibrils are quantified (e.g., a number of fibers, density of fibers, a measure of fiber length, a measure of fiber thickness, measure of branching, a measure of cross-linking, a measure of fiber contrast) using the data corresponding to the detected fluorescent light.

The computer programs can be executed by the computer to implement a method according to one or more embodiments of the present invention whereby interferometric measurements can be made. The computer programs can control the illumination source 201 comprising one (or more) LED that can be used to illuminate layered substrate. The optical path difference (OPD) between the bottom and top surface causes an interference pattern. The interference patterns can be imaged as intensity variations by the CCD camera 232 across the whole substrate at once.

A variety of software programs and formats can be used to store and/or process optical information obtained via the systems and methods described herein. Any number of data processor structuring formats (e.g., text file, database) can be utilized. By providing optical information in computer-readable form, one can use the optical information in readable form to compare a specific optical profile with the optical information stored within a database of the comparison module. For example, direct comparison of the determined optical information from a given sample can be compared to the control data optical information (e.g., data obtained from a control sample). The comparison made in computer-readable form being the retrieved content from the comparison module, which can be processed by a variety of means.

In another embodiment, each incoherent light source can be an optical fiber (not shown) that directs the light at the layered substrate 222. Optical components can be provided to condition the light to uniformly illuminate substantially the entire surface of the layered substrate 222.

In certain embodiments, the reflections from the different layers including the silicon surface (Si) and the silicon dioxide surface (SiO₂) interfere with the light reflected from the fibrils on the surface of the substrate. The interference causes a change in the reflected light, which can be detected by the imaging system as described herein. In certain embodiments, a reflectance signature of the incident light is altered by said fibrils in a layer on the substrate surface to interfere with the light reflected from the silicon surface and the silicon dioxide surface. The imaging system of FIG. 2 detects the interference in the reflection from the fibrils as compared to reflective properties of the silicon surface and the silicon dioxide and an image processing system comprises a forward model to provide accurate and/or quantitative measures of fibrils. An embodiment of the imaging device uses a single wavelength (band) of light to measure the interference/mixing of reflected light from the layer to which the fibrils are bound with the scattered light from the fibril (scattering of the light).

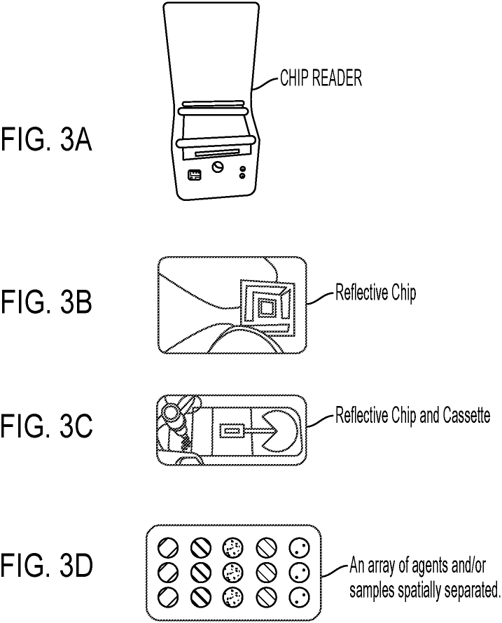

FIG. 3A is an illustrative embodiment of an instrument for the imaging optical substrates as described herein, e.g., for detection of fibrils. FIG. 3B is an image of a reflective chip (substrate), as described herein. FIG. 3C is an image of a reflective chip disposed within a microfluidic cassette, which allows flowing of a sample and/or reagent (e.g., containing a nucleating agent, containing a cross-linking agent) over a substrate. FIG. 3D is an illustration of an array of samples, nucleation agents, capture agents (e.g., antibodies), and/or clotting agents on the substrate, as described herein.

In some embodiments of the exemplified instrument used to image the fibrils, three or more LEDs with different emission peak wavelengths can be used as the light and/or excitation source. In some embodiments where more than one incoherent light source is used, the light sources used have a narrow range of wavelengths, and the width between the wavelengths of each individual light source is small. In some embodiments, the light source may also serve as an excitation source for the excitation of fluorescent probes attached to fibrils. In some embodiments, multiple light sources may be used. In some embodiments, one or more of the light sources is a laser light source.

The use of high-magnification interferometric measurements is an approach to detection of biomolecular targets and fibrils. The methods and devices described herein provide for imaging of such fibrils through the use of a high magnification objective lens with a high numerical aperture and placing a spatial filter on the camera’s optical axis. The high numerical aperture objective lens allows imaging at high magnifications and the spatial filter is used to maintain the contrast of the interference cause by the layered substrate by only collecting light from a high angle or a range of angles of incident light. The optical setup described allows for detection of sub-wavelength structures (e.g., of the fibrils) without losing contrast or lateral resolution.

Another approach to simplifying the imaging device described herein can be to use a broadband source and a colored CCD camera in which the spectral sampling is done by the camera. Pixels of the camera dedicated for detection of separate colors can be used to extract the intensity of light included in a given spectral band, thus allowing a spectral detection scheme of various wavelengths.

One advantage to the embodiments with an LED light source is that an LED based illumination source allows the imaging device to be more robust and portable, thus allowing field applications. Another advantage is that the light source may serve as an excitation source for a fluorophore species that may be excited at a particular wavelength (band) of light. Moreover, the use of multiple LEDs would allow for the simultaneous or sequential excitation of fluorophores. Another advantage is the high magnification capability of the device. High magnification allows for the detection of single fibrils and/or detailed fibril structures. In some embodiments, a white light source or an RGB LED with a 3CCD or other color camera can be used to capture spectral information at three distinct wavelengths to increase temporal resolution. This is beneficial in studying dynamic biological interactions, for example, such as the formation and/or removal of fibrils from a substrate.

The device as described herein facilitates a method of using an LED illumination source for substrate enhanced detection of fibrils in a sample bound to a surface. The LED illumination source may also serve as an excitation source for the detection of fluorescently labeled fibrils. The device provides a high-throughput spectroscopy method for simultaneously recording a response of an entire substrate surface. The device and methods can be used in any high-throughput application. The device and methods thus provide a platform or a system for high-throughput optical sensing of fibrils bound to and/or located substantially close to the surface of a reflective substrate as described herein. The system comprises an illumination source, a reflective substrate, and an imaging device.

In some embodiments, the imaging device comprises a camera. For example, the device can be used for multiplexed and dynamic detection of fibrils. Moreover, in some embodiments, the fibrils may be labeled with or contain a fluorescent probe (tag) to enhance detection.

Certain embodiments of the device can be described as functional modules, which include computer executable instructions recorded on computer readable media and which cause a computer to perform method steps when executed. The modules can be segregated by function for the sake of clarity. However, it should be understood that the modules need not correspond to discrete blocks of code and the described functions can be carried out by the execution of various code portions stored on various media and executed at various times.

In some embodiments, the device provides a system for detecting and/or classifying fibrils on a reflective substrate comprising a) a determination module configured to determine optical information, wherein the optical information comprises sampling a least one wavelength using a narrow band light source; b) a storage device configured to store data output from the determination module; c) a comparison module adapted to compare the data stored on the storage device with a control data, the comparison being a retrieved content; and d) a display module for displaying a page of the retrieved content for the user on the client computer, wherein the retrieved content is a light absorption profile of the substrate, wherein a certain light absorption profile is indicative of the formation of one or more fibrils.

In some embodiments, the imaging device as described herein provides a computer program comprising a computer readable media or memory having computer readable instructions recorded thereon to define software modules including a determination module and a comparison module for implementing a method on a computer, said method comprising a) determining with the determination module optical information, wherein the optical information comprises sampling at least one wavelength using a narrow-band light source; b) storing data output from the determination module; c) comparing with the comparison module the data stored on the storage device with a control data, the comparison being a retrieved content, and d) displaying a page of the retrieved content for the user on the client computer, wherein the retrieved content is a light absorption profile of the solid substrate, wherein a certain light absorption profile is indicative of the formation of one or more fibrils.

Various modules for determining optical properties include, for example, but are not limited to, microscopes, cameras, interferometers (for measuring the interference properties of light waves), photometers (for measuring light intensity); polarimeters (for measuring dispersion or rotation of polarized light), reflectometers (for measuring the reflectance of a surface or object), refractometers (for measuring refractive index of various materials), spectrometers or monochromators (for generating or measuring a portion of the optical spectrum, for the purpose of chemical or material analysis), autocollimators (used to measure angular deflections), and vertometers (used to determine refractive power of lenses such as glasses, contact lenses and magnifier lens).

As used herein, a cassette is defined as configured to contain a reflective substrate as described herein with a transparent and high-quality imaging window (COP or polycarbonate) with a thin channel of fluid.

### B. Applications of the Sensors and Methods

In certain embodiments, the technology relates to methods for the visualization of fibril formation from complex biological samples using a microscopy approach based on interference reflectance (e.g., for clinical applications). The technology allows a user to analyze the temporal (e.g., time of formation, e.g., time of removal) and physical (e.g., density, thickness, branching structures) features of fibers. Described herein are rapid, sensitive, simple to use, and inexpensive biosensors that are useful for a variety of applications involving the detection of fibrils, ranging from research and medical diagnostics.

Accordingly, in certain embodiments, the substrates described herein are used to assess blood clotting and visualization of fibrils (e.g., fibrin fibrils) on a substrate. The presence of fibrils on a substrate layer changes an optical path length relative to an optical path length in the absence of the fibrils, resulting in an interference pattern that is detected and measured by the device and methods described herein. In some embodiments, a sample that contacts the substrate can have a plurality of samples, capture agents, clotting agents and/or nucleation agents. In certain embodiments, a sample that contacts the substrates is and/or contains a complex biological sample (e.g., blood, plasma, saliva) and/or a processed sample (e.g., fibrinogen in a buffer, e.g., a clotting and/or nucleation agent in a buffer).

The devices and substrates can be used to study one or a number of interactions in parallel, i.e., multiplex applications. The substrate is illuminated with light, and if fibrils form on one or more targets on the substrate, the fibrils appear in the image as bright lines (e.g., regions of high contrast). In embodiments where a substrate surface comprises an array of one or more distinct target locations comprising one or more specific clotting and/or nucleation agents, then the interference pattern is detected from each distinct location of the substrate. In certain embodiments, fibrils are then labeled using a fluorescent label to identify the presence and/or absence of fibrils. In certain embodiments, fibril forming components and/or precursors are fluorescently labeled prior to contacting a sample to the substrate.

In some embodiments, a variety of specific samples (e.g., samples containing fibrils and/or fibril precursors), capture agents, clotting agents and/or nucleation agents can be immobilized in an array format onto the substrate surface. The substrate is then contacted with a test sample of interest comprising potential fiber/fibril forming targets, such as fibrin. Only fibrils that form when in contact with a particular combination of a capture agent, a clotting agent and/or a nucleation agent can be able to be visualized on the substrate.

For example, the clotting and/or nucleation agents can be immobilized on a layered substrate surface that has a spectral reflectance signature that is altered upon the formation of fibrils on the substrate surface. In particular, as is described herein, the image processing system detects fibril formation a function of the change in reflective properties of the substrate and an image processing system comprises a model to provide accurate and quantitative measures of the fibrils (e.g., the fibril width, number of fibrils, length of fibrils, branching, etc.). In certain embodiments, changes in these measures may be tracked with time. For example, an embodiment of the device uses a single wavelength (band) of light to measure the interference/mixing of reflected light from the layer on which the fibrils form with the scattered light from the fibril (scattering of the light). As fibrils form on the surface of the substrate, the scattered light from these objects interfere with the reflected light from the substrate surface making the fibrils observable on an imaging device as discrete objects (e.g., dots, lines). The substrate is illuminated with one (or more) wavelengths of light, and if one or more fibrils are formed on a layer, the fibrils can appear in the image as discrete objects, thereby allowing the detection of the formation of fibrils on the substrate as well as the quantitative sizing of the fibrils. In certain embodiments, removal of fibrils from the substrate is tracked. Contacting the surface of the substrate with anti-clotting agents [e.g., blockers of thrombin activation (e.g., thrombomodulin); e.g., modifiers of plasminogen activity such as PAI1 or other serpin molecules; e.g., agents such as warfarin, and factor X inhibitors]. The apparatus allows for the simultaneous imaging of the entire field of view of a surface for high-throughput applications. The apparatus and method has several advantages such as low-cost, high-throughput, rapid and portable detection.

In some respects, the devices and methods described herein provide a high-throughput method for simultaneously recording a response of an entire substrate surface, comprising sampling at least one wavelength using a light source providing incoherent light, and imaging the reflected or transmitted light using an imaging device. The device can include a light-emitting diode (LEDs) as the illumination source for interferometric principles of detection. Interferometric measurements can provide desired sensitivity and resolution using optical path length differences (OPD).

Accordingly, described herein are devices and methods for substrate enhanced detection of fibril formation on a surface of a substrate. The device samples the reflectance spectrum by illuminating the substrate with at least one wavelength of light, using, for example, LEDs and recording the reflectance by an imaging device, such as a 2-D arrayed pixel camera. In this way, the reflectance spectrum for the whole field-of-view is recorded simultaneously. Using this device and method, high-throughput microarray imaging can be accomplished.

The instrument and process provide a high-throughput spectroscopy technique where sampling at least one wavelength is realized by using a narrowband light sources, such as an LED, and the reflected or transmitted light is imaged to an imaging device, such as a monochromatic CCD camera, thus allowing the response of the entire imaged surface to be recorded simultaneously. A microarray can be fabricated on a layered substrate (for example: anywhere from a few nm of SiO₂ up to 100 nm of SiO₂ layered on a Si wafer). An embodiment includes a green LED light source (535 nm) and 100 nm oxide of SiO₂ layered on a Si wafer. A second embodiment includes an ultraviolet LED light source (420 nm) and 60 nm oxide of SiO₂ layered on a Si wafer. A third embodiment, for use when imaging in complex media, includes an ultraviolet LED light source (420 nm) and 30-to-60 nm oxide of SiO₂ layered on a Si wafer.

In some embodiments, three or more LEDs with different emission peak wavelengths can be used as the light source. In some embodiments where more than one incoherent light source is used, the light sources used have a narrow range of wavelength, and the width between the wavelengths of each individual light source is small. In some embodiments, one or two light sources are used.

In some embodiments described herein, the microarray or binding agent is fabricated on a layered substrate comprising anywhere from a few nanometers to 100 nm of SiO₂ layered on a Si wafer. In some embodiments, the microarray or binding agent is fabricated on a layered substrate comprising 95-100 nm of SiO₂ layered on a Si wafer. In some embodiments, the microarray or binding agent is fabricated on a layered substrate comprising 30-60 nm of SiO₂ layered on a Si wafer. An embodiment includes a green LED light source (near 535 nm) and 100 nm oxide of SiO₂ layered on a Si wafer. A second embodiment includes an ultraviolet LED light source (near 420 nm) and 60 nm oxide of SiO₂ layered on a Si wafer. A third embodiment, for use when imaging in complex media, includes an ultraviolet LED light source (near 420 nm) and 30-to-60 nm oxide of SiO₂ layered on a Si wafer. The devices and methods described herein, can be used, in part, for high magnification interferometric measurements, for example, but not limited to, detecting fibrin fibers.

Examples of sensors and methods that can be used with the described optical substrates include, but are not limited to, are described by Daaboul et al., in International Publication No. WO2017/136676 titled “DETECTION OF EXOSOMES HAVING SURFACE MARKERS”, filed on Feb. 3, 2017, the contents of which are hereby incorporated by reference in their entirety.

### I. Imaging and Tracking Fibrin Formation

In certain embodiments, the technology is used to image and track fibrin fibril formation (e.g., natural fibrin formation; e.g., synthetic fibrin formation) via interaction of a test sample with a clotting agent (e.g., an immobilized clotting agent).

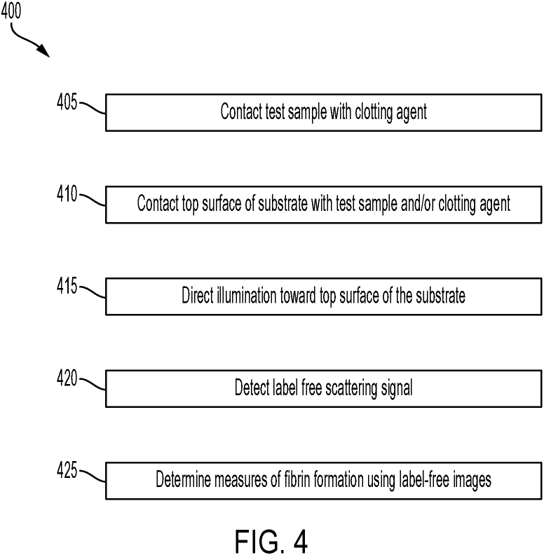

FIG. 4 is a block diagram of a method 400 of imaging and tracking fibrin formation via interaction of a test sample with a clotting agent, according to an illustrative embodiment. In step 405 of method 400, the method comprises contacting a test sample with the clotting agent, so as to combine the test sample and the clotting agent.

In step 410 of method 400, the top surface of the substrate is contacted with the test sample and/or the clotting agent, thereby providing for formation of fibrin at the top surface of the substrate.

In certain embodiments, step 410 may be performed before step 405. For example, the test sample is contacted with the top surface of the substrate prior contacting the test sample with the nucleation agent, and then contacting the top surface of the substrate with the clotting agent. In another example, the top surface of the substrate is contacted with the clotting agent, and then with the test sample. In another example, the clotting agent is contacted with the test sample (e.g., in solution), and then the clotting agent and the test sample are contacted with the top surface of the substrate.

In step 415 of method 400, illumination light is then directed towards the top of the substrate, thereby illuminating the top surface of the substrate along with the fibrin formed thereon.

In step 420 of method 400, one or more imaging detectors are used to detect a label-free scattering signal corresponding to a portion of the illumination light that is (A) scattered by the fibrin (e.g., fibrin fibrils), and/or (B) reflected by the reflective substrate, thereby obtaining one or more label-free images of fibrin formation (e.g., fibrin fibril formation).

In step 425 of method 400, the images are then used to determine one or more measures of fibrin fibril formation. In certain embodiments, these measures of fibrin formation may be, for example, one or more static measures (e.g., average density, mass, branching, cross-linking, a number of fibers formed, a measure of fiber length, a measure of fiber contrast; a measure fiber width, etc.). In certain embodiments, associated with a point in time, a series of points in time, and/or a label-free image.

### II. Tracking Fibrin Removal

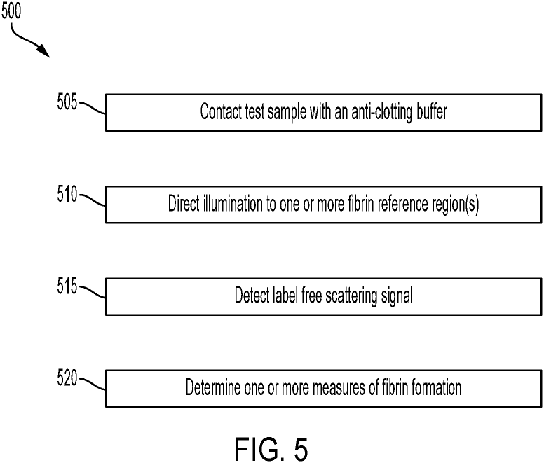

In certain embodiments, the technology is used to image and track fibrin removal by an anti-clotting agent. FIG. 5 is a block diagram of method 500 of imaging and tracking fibrin (e.g., fibrin fibril) removal by an anti-clotting agent, according to an illustrative embodiment.

In step 505 of method 500, the top surface of a substrate with an anti-clotting buffer comprising an anti-clotting agent.

In step 510 of method 500, the illumination light is directed toward at least a portion of the one or more fibrin reference regions of the top surface of the substrate, thereby illuminating the top surface of the substrate along with fibrin formed thereon within the portion of the fibrin reference regions.

In step 515 of method 500, one or more imaging detectors is then used to detect a label-free scattering signal corresponding to a portion of the illumination light that is (A) scattered the fibrin, and/or (B) reflected by the reflective substrate, thereby obtaining one or more label-free images of fibrin formation (e.g., fibrin fibril formation).

In step 520 of method 500, the detected label-free scattering signal is then used to determine one or more measures of fibrin formation (e.g., average density, mass, branching, etc.) for the test sample. In certain embodiments, the one or more measures of fibrin formation are then used to determine one or more prognostic values indicative of thrombotic risk, clotting characteristics, disease state, within the patient.

### III. Immobilization of a Clotting Agent on a Surface for Generating Fibrin Formation

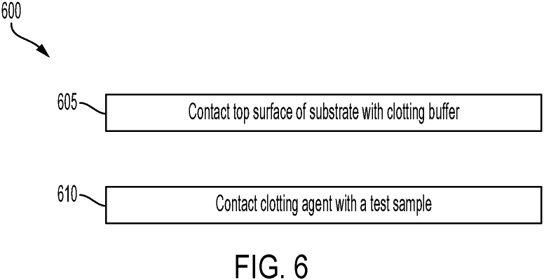

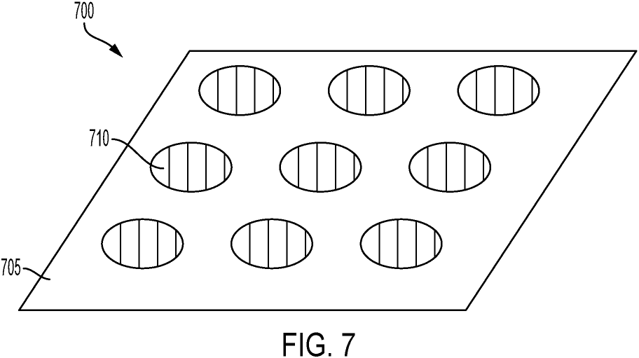

In certain embodiments, the technology may be used to generating fibrin formation on a surface of a substrate (e.g., a substantially planar substrate) for chip-based testing via immobilized clotting agents (e.g., thrombin). FIG. 6 is a block diagram of a method 600 of generating fibrin formation on a surface of a substrate for chip-based testing via immobilized clotting agents. An illustrative embodiment of the substrate 700 is shown in FIG. 7.

In step 605 of method 600, a top surface of the substrate is contacted with a clotting buffer comprising a clotting agent wherein the top surface of the substrate 705 (FIG. 7) comprises one or more capture agents 710, each capture agent specific to the particular clotting agent, thereby capturing the clotting agent onto the top surface of the substrate. In certain embodiments, the capture agent may be fibrinogen, collagen, or a capture agent (e.g., an antibody) specific to fibrinogen.

Following step 605, step 610 of method 600 the clotting agent is contacted with a test sample.

### IV. Imaging and Tracking Fibril Formation

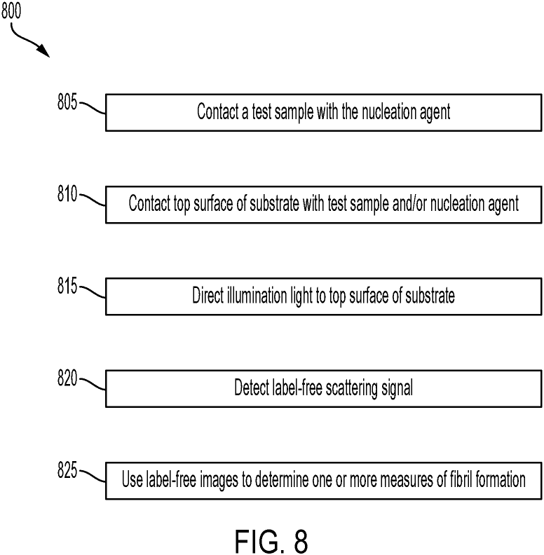

In certain embodiments, the technology is used to image and track fibril (e.g., fibrin fibril) formation via interaction of a test sample with a nucleation agent. FIG. 8 is a block diagram of a method 800 of imaging and tracking fibril formation via interaction of a test sample with a nucleation agent.

In step 805 of method 800, a test sample is contacted with the nucleation agent. In step 810 of method 800, a top surface of a substrate is contacted with (i) the test sample and/or (ii) the nucleation agent, thereby providing for formation of one or more fibrils at the top surface of the substrate.

In certain embodiments, step 810 may be performed before step 805. For example, the test sample is contacted with the top surface of the substrate prior contacting the test sample with the nucleation agent, and then contacting the top surface of the substrate with the nucleation agent. In another example, the top surface of the substrate is contacted with the nucleation agent, and then with the test sample. In another example, the nucleation agent is contacted with the test sample (e.g., in solution), and then the nucleation agent and the test sample are contacted with the top surface of the substrate.

In step 815 of method 800, the illumination light is directed toward the top surface of the substrate, thereby illuminating the top surface of the substrate along with the fibril formed thereon.

In step 820 of method 800, one or more imaging detectors is used to detect a label-free scattering signal corresponding to a portion of the illumination light that is (A) scattered by the fibril, and/or (B) reflected by the reflective substrate, thereby obtaining one or more label-free images of fibril formation.

In step 825 of method 800, one or more label-free images are used to determine one or more measures of fibril formation.

It is understood that the foregoing detailed description and the following examples are illustrative only and are not to be taken as limitations upon the scope of the invention. Various changes and modifications to the disclosed embodiments, which will be apparent to those, skilled in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the Applicants and do not constitute any admission as to the correctness of the dates or contents of these documents.

### C. Example

### I. Experimental Results Demonstrating the Presence of Fibrinogen

In certain embodiments, the systems and methods described herein can be used to assess the formation of fibrin from a sample solution containing fibrinogen. In this example, an embodiment of the systems and methods described herein uses a light-microscopy approach based on interference-reflectance microscopy to directly observe the formation of fibrin on a substrate. The substrate was scanned and imaged using the IRIS (Interferometric Reflectance Imaging Sensor) system, a commercially available device from NanoView Biosciences, Inc. that images a silicon substrate with a thin silicon dioxide top layer comprising spots and/or regions of interest. In certain embodiments, a system such as the one described in in PCT/US17/16434 entitled “DETECTION OF EXOSOMES HAVING SURFACE MARKERS” filed on Feb. 3, 2017, the content of which is hereby incorporated by reference in its entirety, is used. The present example provides direct evidence of fibrin formation (e.g., via the formation of fibrin fibrils) on a substrate and to definitively prove the structures contain fibrinogen.

In this example, an 8 x 8 arrangement of fibrinogen spots were created using a robotic spotter on top of the substrate using fibrinogen of varying concentrations in a solution of phosphate buffered saline (PBS; 8 g/L NaCl; 0.2 g/L KC1; 1.44 g/L Na₂HPO₄, 0.24 g/L KH₂PO₄ in water) with 25 mM of trehalose. The concentrations of the spotted protein locations consisted of 8 rows of the following fibrinogen concentrations: 3.7 mg/mL, 0.75 mg/mL, 0.075 mg/mL, 7.5E-3mg/mL, 7.5E-4mg/mL, 7.5E-5mg/mL, 7.5E-6mg/mL, and 7.5E-7mg/mL. After spotting, the chips were washed for 15 min in PBS with 0.1% Tween®-20 (PBST) for 15 min and stored at room temperature until experimentation. It should be noted that Triton™ may also be substituted for Tween®.

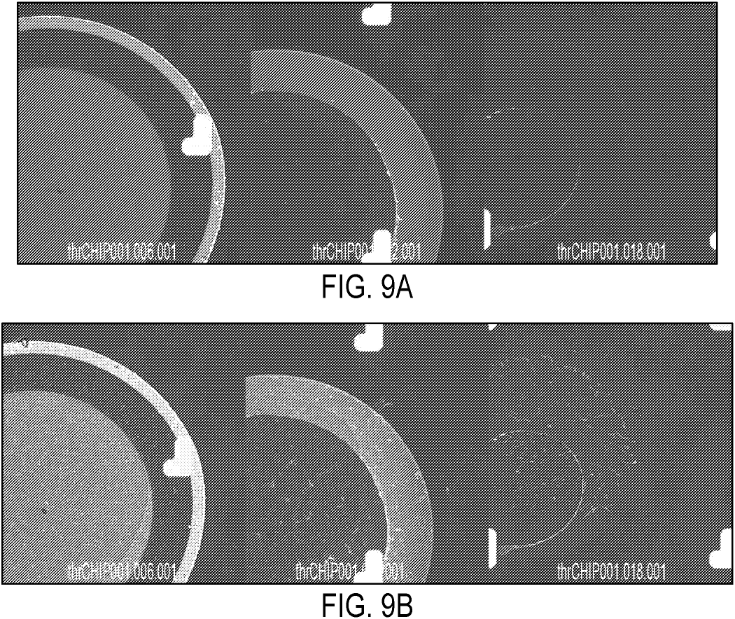

Chips were then pre-scanned (or imaged before contact with a clotting agent such as thrombin solution to activate fibrinogen) using IRIS to acquire an image. FIG. 9A shows a series of images of a fibrinogen spotted, pre-scanned chip prior to incubation with thrombin. The concentration of the fibrinogen spots decreases from left to right and are listed as follows: 3.7 mg/mL, 0.75 mg/mL, and 0.075 mg/mL. The chips were then incubated for 3 min on an orbital shaker with 30µL of 2 nM alpha-thrombin in a solution of tris-buffered saline (TBS) with 1% gelatin (TBSG) to “activate” the fibrinogen spots. Then, 30µL of high-concentration fibrinogen in TBSG was added to the activated chip incubated for 3 min. Chips were then washed with 3 times with PBS for a 3 min duration for each wash. Finally, the chips were washed in deionized, distilled water (ddH₂O) and then scanned and imaged again using IRIS. FIG. 9B shows an image of the formation of fibrin fibers onto the surface of the same exact post-scanned chip spots (e.g., the image of the leftmost spot is the same spot as previously presented in the leftmost spot, but with fibrin fibers forming on the surface) of FIG. 9A.

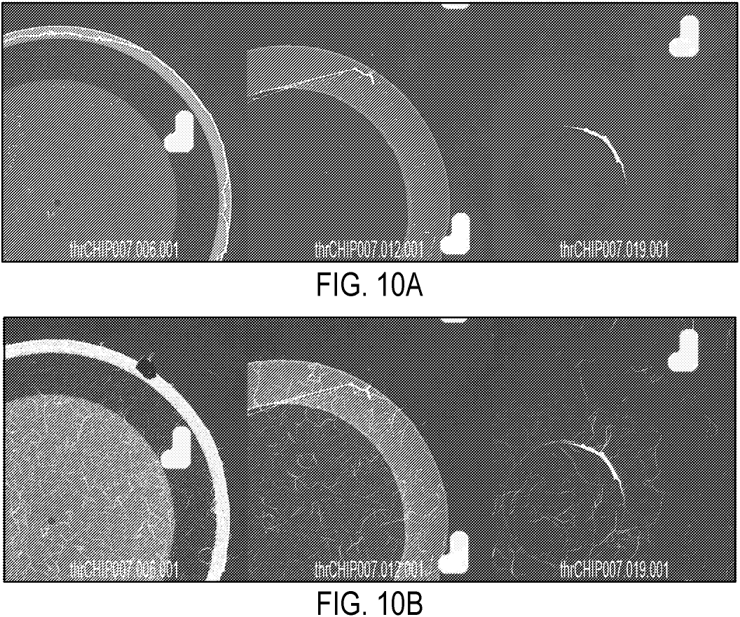

A second chip was created using the same spotting procedure as before. New images were taken as described previously. FIG. 10A is same as FIG. 9A, but on a new chip. As before, the concentration of the fibrinogen spots decreases from left to right in the images: 3.7 mg/mL, 0.75 mg/mL, and 0.075 mg/mL. The same activation step was carried out as before. Next, a sample of fibrinogen with the same composition as before was incubated on the surface of the chip for 10 min instead of 3 min. This was then followed by the same washing, scanning, and imaging steps. Images of the same spots taken after scanning are presented in FIG. 10B. Fibrin fibers can again be seen forming bright lines on the surface of the substrate. These fibrin fibers are longer than those appearing when the incubation was only 3 min (FIG. 9B).

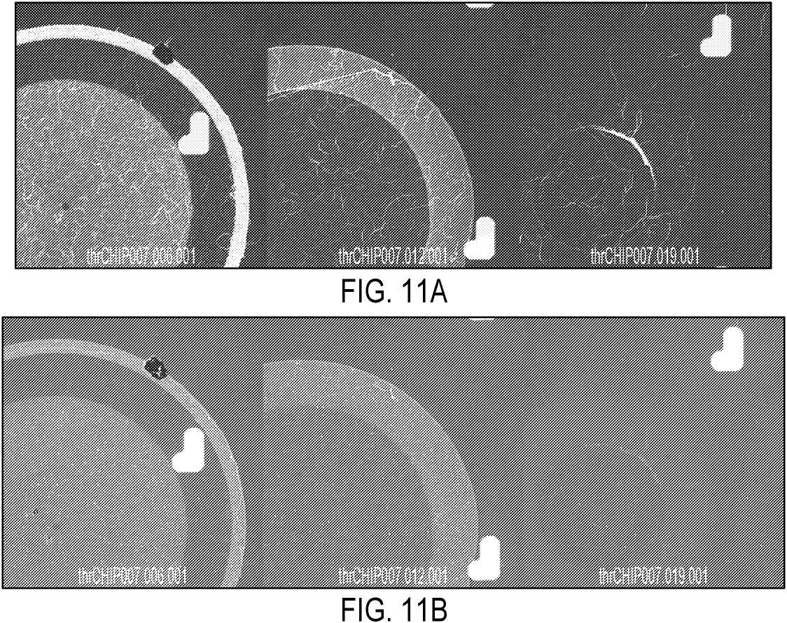

In order to demonstrate that the fibers forming on the surface were indeed fibrin, plasmin, an enzyme that digests fibrin, was incubated on spots of the substrate where fibrin fibers had been formed. FIG. 11A is the same series of images as FIG. 10B. The series of images show bright lines indicating fibrin fibers forming branching and elongated structures on the surface of the microarray substrate. This image was taken prior to incubation with plasmin. FIG. 11B shows three images of the same exact spots of FIG. 11A after they have been incubated with plasmin. Much of the branching structures and fibers have been digested as can be seen by their disappearance from the images.

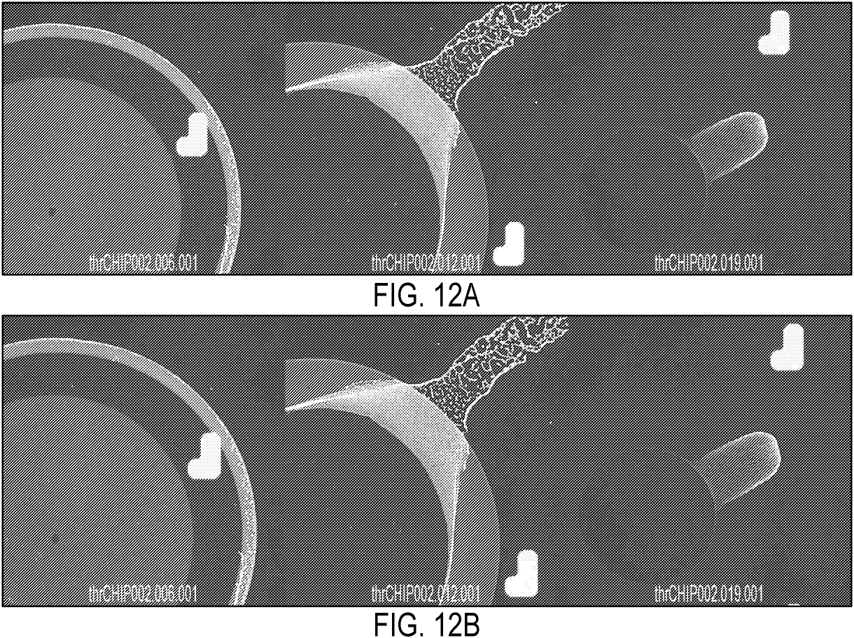

To further demonstrate that the fibrous structures on the chips are indeed fibrin from the solution and not simply from the chip, a chip was treated in the same manner as before; however, no fibrinogen was present in the solution incubated on the spots. As before, the chip was created using the same procedure to deposit fibrin spots of varying concentrations. The series of images of FIG. 12A are a pre-scanned spots on a substrate using the same preparation procedures as FIGS. 9A and 10A. As before, the concentration of the fibrinogen of spots decreases from left to right: 3.7 mg/mL, 0.75 mg/mL, and 0.075 mg/mL. However, instead of incubating a solution of high concentration fibrinogen in TBSG on each of the spotted protein areas, TBSG alone was incubated on the fibrinogen spots for 10 min. The same washing procedures were carried out as before. Images of the spots were then taken after scanning (FIG. 12B). The images demonstrate that there is no fiber formation when no fibrinogen is present in the solution, thus demonstrating the need for fibrinogen to be present in solution to generate fibrin fibers on the surface.

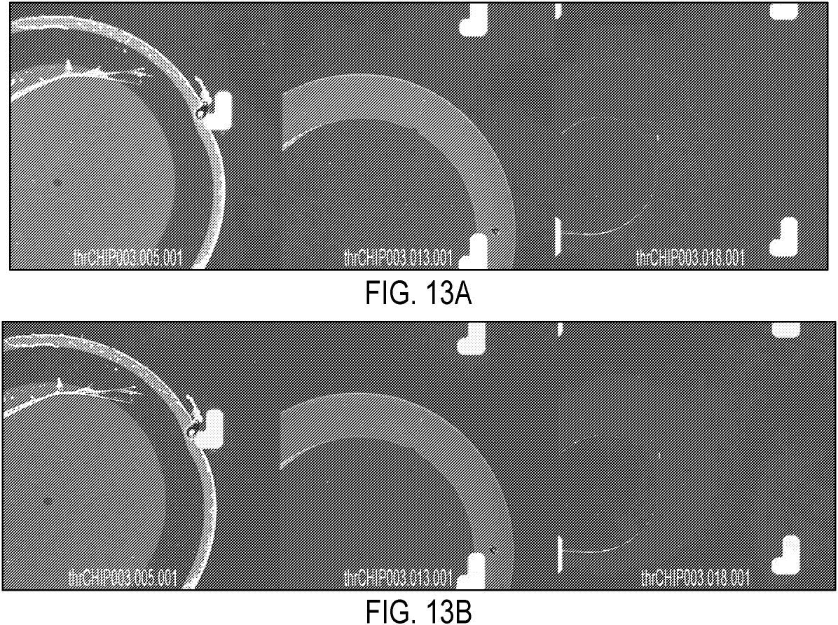

To further demonstrate that the formation of fibrin fibers requires alpha-thrombin, the same procedure was carried out for the preparation of the substrate as in FIGS. 9A and 10A to produce FIG. 13A. The concentration of the fibrinogen spots in the images of FIG. 13A decreases from left to right: 3.7 mg/mL, 0.75 mg/mL, and 0.075 mg/mL. The chips were then incubated for 3 min on an orbital shaker with 30µL of a solution of TBSG. This step differs from the previous experiments in that this solution lacks alpha-thrombin to activate the spots. Then, 30µL of high-concentration fibrinogen in TBSG was added to the chip and incubated for 10 min. The same washing procedure was carried out as before. The chips were then scanned and imaged. FIG. 13B shows a series of images of the same spots on the post-scanned substrate with no fibers forming, thereby demonstrating that alpha-thrombin was needed for the formation of fibrin fibers on the substrate.

What is shown by this experiment is that by using spotted microarrays of a range of concentrations of fibrinogen, IRIS can be used to directly observe the formation and removal of fibrin fibers on the surface of a substrate, which has far reaching implications regarding the diagnostic capabilities of such a technology.

### D. Computer System and Network Environment

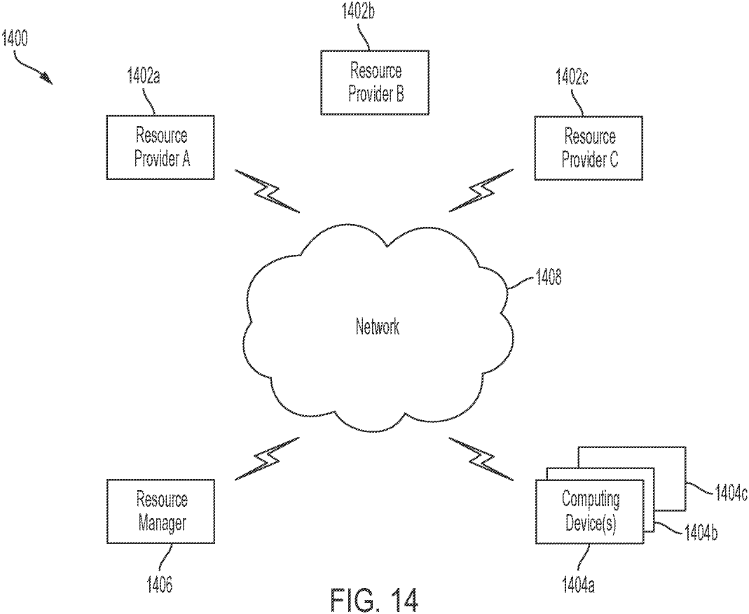

As shown in FIG. 14, an implementation of a network environment 1400 for use in providing the systems and methods described herein is shown and described. In brief overview, referring now to FIG. 14, a block diagram of an exemplary cloud computing environment 1400 is shown and described. The cloud computing environment 1400 may include one or more resource providers 1402a, 1402b, 1402c (collectively, 1402). Each resource provider 1402 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 1402 may be connected to any other resource provider 1402 in the cloud computing environment 1400. In some implementations, the resource providers 1402 may be connected over a computer network 1408. Each resource provider 1402 may be connected to one or more computing device 1404a, 1404b, 1404c (collectively, 1404), over the computer network 1408.

The cloud computing environment 1400 may include a resource manager 1406. The resource manager 1406 may be connected to the resource providers 1402 and the computing devices 1404 over the computer network 1408. In some implementations, the resource manager 1406 may facilitate the provision of computing resources by one or more resource providers 1402 to one or more computing devices 1404. The resource manager 1406 may receive a request for a computing resource from a particular computing device 1404. The resource manager 1406 may identify one or more resource providers 1402 capable of providing the computing resource requested by the computing device 1404. The resource manager 1406 may select a resource provider 1402 to provide the computing resource. The resource manager 1406 may facilitate a connection between the resource provider 1402 and a particular computing device 1404. In some implementations, the resource manager 1406 may establish a connection between a particular resource provider 1402 and a particular computing device 1404. In some implementations, the resource manager 1406 may redirect a particular computing device 1404 to a particular resource provider 1402 with the requested computing resource.

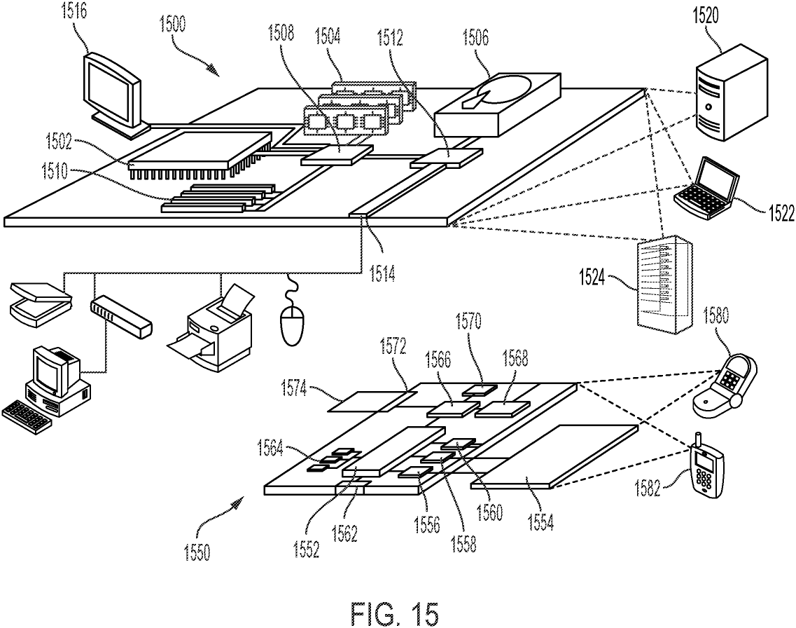

FIG. 15 shows an example of a computing device 1500 and a mobile computing device 1550 that can be used to implement the techniques described in this disclosure. The computing device 1500 is intended to represent various forms of digital computers, such as laptops, desktops, workstations, personal digital assistants, servers, blade servers, mainframes, and other appropriate computers. The mobile computing device 1550 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 1500 includes a processor 1502, a memory 1504, a storage device 1506, a high-speed interface 1508 connecting to the memory 1504 and multiple high-speed expansion ports 1510, and a low-speed interface 1512 connecting to a low-speed expansion port 1514 and the storage device 1506. Each of the processor 1502, the memory 1504, the storage device 1506, the high-speed interface 1508, the high-speed expansion ports 1510, and the low-speed interface 1512, are interconnected using various busses, and may be mounted on a common motherboard or in other manners as appropriate. The processor 1502 can process instructions for execution within the computing device 1500, including instructions stored in the memory 1504 or on the storage device 1506 to display graphical information for a GUI on an external input/output device, such as a display 1516 coupled to the high-speed interface 1508. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system). Thus, as the term is used herein, where a plurality of functions are described as being performed by “a processor”, this encompasses embodiments wherein the plurality of functions are performed by any number of processors (one or more) of any number of computing devices (one or more). Furthermore, where a function is described as being performed by “a processor”, this encompasses embodiments wherein the function is performed by any number of processors (one or more) of any number of computing devices (one or more) (e.g., in a distributed computing system).

The memory 1504 stores information within the computing device 1500. In some implementations, the memory 1504 is a volatile memory unit or units. In some implementations, the memory 1504 is a non-volatile memory unit or units. The memory 1504 may also be another form of computer-readable medium, such as a magnetic or optical disk.

The storage device 1506 is capable of providing mass storage for the computing device 1500. In some implementations, the storage device 1506 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 1502), perform one or more methods, such as those described above. The instructions can also be stored by one or more storage devices such as computer- or machine-readable mediums (for example, the memory 1504, the storage device 1506, or memory on the processor 1502).

The high-speed interface 1508 manages bandwidth-intensive operations for the computing device 1500, while the low-speed interface 1512 manages lower bandwidth-intensive operations. Such allocation of functions is an example only. In some implementations, the high-speed interface 1508 is coupled to the memory 1504, the display 1516 (e.g., through a graphics processor or accelerator), and to the high-speed expansion ports 1510, which may accept various expansion cards (not shown). In the implementation, the low-speed interface 1512 is coupled to the storage device 1506 and the low-speed expansion port 1514. The low-speed expansion port 1514, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 1500 may be implemented in a number of different forms, as shown in the figure. For example, it may be implemented as a standard server 1520, or multiple times in a group of such servers. In addition, it may be implemented in a personal computer such as a laptop computer 1522. It may also be implemented as part of a rack server system 1524. Alternatively, components from the computing device 1500 may be combined with other components in a mobile device (not shown), such as a mobile computing device 1550. Each of such devices may contain one or more of the computing device 1500 and the mobile computing device 1550, and an entire system may be made up of multiple computing devices communicating with each other.

The mobile computing device 1550 includes a processor 1552, a memory 1564, an input/output device such as a display 1554, a communication interface 1566, and a transceiver 1568, among other components. The mobile computing device 1550 may also be provided with a storage device, such as a micro-drive or other device, to provide additional storage. Each of the processor 1552, the memory 1564, the display 1554, the communication interface 1566, and the transceiver 1568, are interconnected using various buses, and several of the components may be mounted on a common motherboard or in other manners as appropriate.

The processor 1552 can execute instructions within the mobile computing device 1550, including instructions stored in the memory 1564. The processor 1552 may be implemented as a chipset of chips that include separate and multiple analog and digital processors. The processor 1552 may provide, for example, for coordination of the other components of the mobile computing device 1550, such as control of user interfaces, applications run by the mobile computing device 1550, and wireless communication by the mobile computing device 1550.

The processor 1552 may communicate with a user through a control interface 1558 and a display interface 1556 coupled to the display 1554. The display 1554 may be, for example, a TFT (Thin-Film-Transistor Liquid Crystal Display) display or an OLED (Organic Light Emitting Diode) display, or other appropriate display technology. The display interface 1556 may comprise appropriate circuitry for driving the display 1554 to present graphical and other information to a user. The control interface 1558 may receive commands from a user and convert them for submission to the processor 1552. In addition, an external interface 1562 may provide communication with the processor 1552, so as to provide for near area communication of the mobile computing device 1550 with other devices. The external interface 1562 may provide, for example, for wired communication in some implementations, or for wireless communication in other implementations, and multiple interfaces may also be used.

The memory 1564 stores information within the mobile computing device 1550. The memory 1564 can be implemented as one or more of a computer-readable medium or media, a volatile memory unit or units, or a non-volatile memory unit or units. An expansion memory 1574 may also be provided and connected to the mobile computing device 1550 through an expansion interface 1572, which may include, for example, a SIMM (Single In Line Memory Module) card interface. The expansion memory 1574 may provide extra storage space for the mobile computing device 1550, or may also store applications or other information for the mobile computing device 1550. Specifically, the expansion memory 1574 may include instructions to carry out or supplement the processes described above, and may include secure information also. Thus, for example, the expansion memory 1574 may be provide as a security module for the mobile computing device 1550, and may be programmed with instructions that permit secure use of the mobile computing device 1550. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier. The instructions, when executed by one or more processing devices (for example, processor 1552), perform one or more methods, such as those described above. The instructions can also be stored by one or more storage devices, such as one or more computer- or machine-readable mediums (for example, the memory 1564, the expansion memory 1574, or memory on the processor 1552). In some implementations, the instructions can be received in a propagated signal, for example, over the transceiver 1568 or the external interface 1562.

The mobile computing device 1550 may communicate wirelessly through the communication interface 1566, which may include digital signal processing circuitry where necessary. The communication interface 1566 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 1568 using a radio-frequency. In addition, short-range communication may occur, such as using a Bluetooth®, Wi-Fi™, or other such transceiver (not shown). In addition, a GPS (Global Positioning System) receiver module 1570 may provide additional navigation- and location-related wireless data to the mobile computing device 1550, which may be used as appropriate by applications running on the mobile computing device 1550.

The mobile computing device 1550 may also communicate audibly using an audio codec 1560, which may receive spoken information from a user and convert it to usable digital information. The audio codec 1560 may likewise generate audible sound for a user, such as through a speaker, e.g., in a handset of the mobile computing device 1550. Such sound may include sound from voice telephone calls, may include recorded sound (e.g., voice messages, music files, etc.) and may also include sound generated by applications operating on the mobile computing device 1550.

The mobile computing device 1550 may be implemented in a number of different forms, as shown in the figure. For example, it may be implemented as a cellular telephone 1580. It may also be implemented as part of a smart-phone 1582, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some implementations, any modules described herein can be separated, combined or incorporated into single or combined modules. Modules depicted in the figures are not intended to limit the systems described herein to the software architectures shown therein.

Elements of different implementations described herein may be combined to form other implementations not specifically set forth above. Elements may be left out of the processes, computer programs, databases, etc. Described herein without adversely affecting their operation. In addition, the logic flows depicted in the figures do not require the particular order shown, or sequential order, to achieve desirable results. Various separate elements may be combined into one or more individual elements to perform the functions described herein.

Throughout the description, where apparatus and systems are described as having, including, or comprising specific components, or where processes and methods are described as having, including, or comprising specific steps, it is contemplated that, additionally, there are apparatus, and systems of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

While the invention has been particularly shown and described with reference to specific preferred embodiments, it should be understood by those skilled in the art that various changes in form and detail may be made therein without departing from the spirit and scope of the invention as defined by the appended claims.

### INCORPORATION BY REFERENCE

All publications and patents mentioned herein are hereby incorporated by reference in their entirety as if each individual publication or patent was specifically and individually indicated to be incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of imaging and tracking fibrin formation via interaction of a test sample with a clotting agent, the method comprising:
(a) contacting the test sample with the clotting agent;
(b) contacting a top surface of a substrate with the (i) the test sample and/or (ii) the clotting agent, thereby providing for formation of fibrin at the top surface of the substrate;
(c) directing illumination light toward the top surface of the substrate, thereby illuminating the top surface of the substrate along with the fibrin formed thereon;
(d) detecting, with one or more imaging detectors, a label-free scattering signal corresponding to a portion of the illumination light that is (A) scattered by the fibrin, and/or (B) reflected by the reflective substrate, thereby obtaining one or more label-free images of fibrin formation; and
(e) using the one or more label-free images to determine one or more measures of fibrin formation.

2-3. (canceled)

4. The method of claim 1, wherein the top surface of the substrate comprises one or more primary capture agents, each specific to at least one of (i) the one or more components of the test sample, (ii) the clotting agent, and (iii) the one or more product components.

5. The method of claim 1, comprising drying the top surface of the substrate following step (b) and before performing step (c).

6. The method of claim 1, wherein step (b) comprises incubating the clotting agent with the test sample for a duration of about 5 minutes or less.

7. The method of claim 1, comprising performing steps (c) and (d) at one or more time points after contacting the clotting agent with the test sample, so as to obtain one or more label-free images of fibrin formation, each corresponding to a particular time point after the contacting the clotting agent with the test sample.

8. The method of claim 7, comprising performing steps (c) and (d) at a plurality of time points while incubating the clotting agent with the test sample, thereby obtaining a plurality of images tracking formation of fibrin.

9. The method of claim 7, comprising performing steps (c) and (d) at one or more times prior to and/or at the same time as step (b), thereby obtaining one or more reference images of the top surface of the substrate prior to formation of fibrin following the contacting the clotting agent with the test sample.

10. The method of claim 1, wherein step (d) comprises imaging the top surface of the substrate and/or any fibers formed thereon at a resolution better than 600 nm.

11. The method of claim 1, wherein the one or more measures of fibrin formation comprise one or more members selected from the group consisting of:
a number of fibers;
a density of fibers;
a measure of fiber length;
a measure of fiber thickness;
a measure of branching capacity;
a measure of fibrin cross-linking; and
a measure of contrast.

12. The method of claim 1, wherein step (e) comprises:
identifying, within at least a portion of the one or more label-free images, one or more point spread functions each corresponding to a piece of fibrin having a sub-diffraction limited length;
determining, for each of the one or more point spread functions, a contrast value, thereby determining one or more contrast values; and
using at least a portion of the one or more determined contrast values to determine a length of the corresponding piece of fibrin.

13. The method of claim 1, comprising performing steps (c) and (d) at a plurality of time points while incubating the clotting agent with the test sample, thereby obtaining a plurality of images tracking formation of fibrin, and using the plurality of images to determine one or more time-dependent measures of fibrin formation.

14. The method of claim 1, comprising using the one or more measures of fibrin formation to determine one or more prognostic values for the test sample and/or a subject associated with the test sample.

15. The method of claim 14, wherein the one or more prognostic values comprise an activated partial thromboplastin time (APPT) and/or a prothrombin time (PT).

16. The method of claim 14, wherein the one or more prognostic values comprises a relative risk of one or more particular diseases and/or conditions.

17. (canceled)

18. The method of claim 1, wherein one or more fibrin forming components is/are fluorescently labeled.

19. (canceled)

20. The method of claim 1, wherein the top surface of the substrate comprises one or more secondary capture agents, each specific to one or more disease-associated biomolecules, each disease-associated biomolecule associated with a particular infectious disease, thereby providing for testing the test sample for the particular infectious disease.

21. The method of claim 1, wherein fibrin-capturing molecules are used to assess the presence of micro-clots within a plasma or blood sample.

22. (canceled)

23. The method of claim 1, wherein the top surface of the substrate comprises a material under test.

24. The method of claim 1, further comprising contacting the test sample with one or more secondary agents, thereby providing for assessing the influence of the one or more secondary agents on clotting and/or assessing removal of clots via the one or more secondary agents.

25. The method of claim 24, wherein the one or more secondary agents comprise anti-clotting agents.

26. The method of claim 24, wherein the one or more secondary agents comprise one or more clot promoting agents.

27-28. (canceled)

29. A method of imaging and tracking fibrin removal by an anti-clotting agent, the method comprising:
(a) contacting a top surface of a substrate with an anti-clotting buffer comprising an anti-clotting agent, wherein the top surface of the substrate comprises one or more fibrin reference regions each comprising a known fibrin layer;
(b) directing illumination light toward at least a portion of the one or more fibrin reference regions of the top surface of the substrate, thereby illuminating the top surface of the substrate along with fibrin formed thereon within the portion of the fibrin reference regions;
(c) detecting, with one or more imaging detectors, a label-free scattering signal corresponding to a portion of the illumination light that is (A) scattered the fibrin, and/or (B) reflected by the reflective substrate, thereby obtaining one or more label-free images of fibrin formation; and
(d) using the detected label-free scattering signal to determine one or more measures of fibrin formation for the test sample.

30-42. (canceled)

43. A system for imaging and tracking fibrin formation via interaction of a test sample with a clotting agent, the system comprising:
(a) a planar reflective substrate comprising one or more capture agents and/or one or more fibrin reference regions;
(b) a mount for holding a substrate;
(c) one or more illumination light sources aligned with respect to the mount so as to and direct illumination light toward a top surface of the substrate, so as to provide for illumination of the top surface of the substrate along with fibrin formed thereon;
(d) one or more detectors aligned with respect to the mount and operable to detect a portion of the illumination light that is (A) scattered by the fibrin, and/or (B) reflected by the reflective substrate, thereby providing for obtaining one or more label-free images of fibrin formation;
(e) a processor of a computing device; and
(f) a memory having instructions stored thereon, wherein the instructions, when executed by the processor, cause the processor to:
receive and/or access data corresponding to the one or more label free images; and
use the one or more label-free images to determine one or more measures of fibrin formation.

44. The system of claim 43, wherein, the top surface of the planar reflective substrate comprises a plurality of capture agent spots, each capture agent spot comprising a particular capture agent specific to a particular clotting agent and/or component of fibrin.

45. The system of claim 43, wherein the top surface of the planar reflective substrate comprises a plurality of fibrin reference regions, each comprising a known fibrin layer.

46. The system of claim 43, comprising an objective lens aligned to (i) collect light (A) scattered by the fibrin and/or reflected by the reflective substrate and (ii) direct the collected light onto the one or more detectors.

47-51. (canceled)
